(19) United States
(12) Patent Application Publication
(10) Pub. No.: US 2025/0302775 A1
(43) Pub. Date: Oct. 2, 2025

(54) METHODS FOR THE TREATMENT OF DEPRESSION

(71) Applicant: Autobahn Therapeutics, Inc., San Diego, CA (US)

(72) Inventors: Kevin FINNEY, Poway, CA (US); Gudarz DAVAR, Princeton, NJ (US); Rohan GANDHI, San Diego, CA (US)

(21) Appl. No.: 19/109,521

(22) PCT Filed: Sep. 8, 2023
(86) PCT No.: PCT/US2023/073777

§ 371 Date: Mar. 6, 2025

Related U.S. Application Data
(60) Provisional application No. 63/375,192, filed on Sep. 9, 2022.

(51) Int. Cl. A61K 31/165; A61K 31/167; A61K 31/277; A61K 31/337; A61K 31/397; A61K 31/40; A61K 31/42; A61K 31/44; A61K 31/4406; A61K 31/4409; A61K 31/455; A61K 31/4965; A61K 31/50; A61K 31/505; A61K 31/5375; A61K 45/06; A61P 25/24

(52) U.S. Cl. CPC A61K 31/165; A61K 31/167; A61K 31/277; A61K 31/337; A61K 31/397; A61K 31/40; A61K 31/42; A61K 31/44; A61K 31/4406; A61K 31/4409; A61K 31/455; A61K 31/4965; A61K 31/50; A61K 31/505; A61K 31/5375; A61K 45/06; A61P 25/24

(57) ABSTRACT

Provided herein are methods for treating depression with a fatty acid amide hydrolase (FAAH) cleavable prodrug of a thyroid hormone receptor (TRβ) agonist.

### CROSS-REFERENCE

This application claims benefit of U.S. Provisional Patent Application No. 63/375,192, filed on Sep. 9, 2022, which is incorporated herein by reference in its entirety.

### BACKGROUND OF THE INVENTION

The blood-brain barrier is composed of tightly linked endothelial cells that limit the passage of pathogens and specific types of small and large molecules from the blood into the brain. This critical protective function also restricts the diffusion of therapeutics into the brain representing a major challenge to the development of new medicines for depression.

### SUMMARY OF THE INVENTION

In one aspect provided herein is a method of treating depression, anxiety disorders, or pain in a patient in need thereof comprising administering to the patient a therapeutically effective amount of a compound of Formula (I), or a pharmaceutically acceptable salt or solvate thereof:

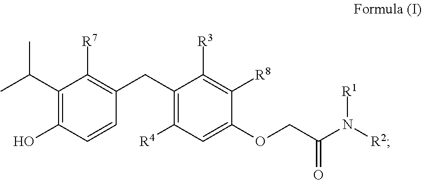

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I;
R⁵ and R⁶ are independently selected from hydrogen and C₁-C₆alkyl; and
R⁷ and R⁸ are independently selected from hydrogen, —F, —Cl, —Br, and —I.

In another aspect provided herein is a method of treating depression, anxiety disorders, or pain in a patient in need thereof comprising administering to the patient a therapeutically effective amount of a compound of Formula (II), or a pharmaceutically acceptable salt or solvate thereof:

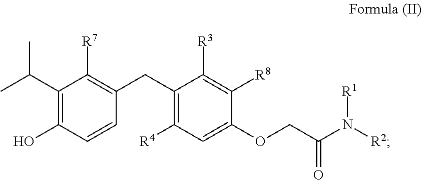

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I;
R⁵ and R⁶ are independently selected from hydrogen and C₁-C₆alkyl; and
R⁷ and R⁸ are independently selected from hydrogen, —F, —Cl, —Br, and —I, wherein at least one of R⁷ and R⁸ is not hydrogen.

In some embodiments, R⁷ is hydrogen. In some embodiments, R⁸ is hydrogen. In some embodiments, R⁸ is —F. In some embodiments, R⁷ is —F.

In another aspect provided herein is a method of treating depression, anxiety disorders, or pain in a patient in need thereof comprising administering to the patient a therapeutically effective amount of a compound of Formula (III), or a pharmaceutically acceptable salt or solvate thereof:

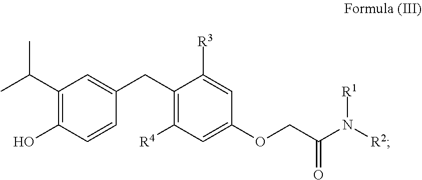

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I; and
R⁵ and R⁶ are independently selected from hydrogen and C₁-C₆alkyl.

In some embodiments, R¹ is hydrogen. In some embodiments, R² is C₁-C₆alkyl optionally substituted with one or more of halo, cyano, —OR⁵, —NR⁵R⁶, —S(O)₂R⁵, or —S(O)₂OR⁵.

In some embodiments, R² is C₁-C₆alkyl substituted with one or more of halo, cyano, —OR⁵, —NR⁵R⁶, —S(O)₂R⁵, or —S(O)₂OR⁵. In some embodiments, R² is C₁-C₆alkyl substituted with one or more —OH. In some embodiments, R² is C₁-C₆alkyl substituted with one or more of halo. In some embodiments, R² is unsubstituted C₁-C₆alkyl. In some embodiments, R² is phenyl optionally substituted with one or more of halo, cyano, —OR⁵, —NR⁵R⁶, —S(O)₂R⁵, or —S(O)₂OR⁵.

In some embodiments, R² is —C₁-C₆alkyl-phenyl optionally substituted with one or more of halo, cyano, —OR⁵, —NR⁵R⁶, —S(O)₂R⁵, or —S(O)₂OR⁵. In some embodiments, R³ and R⁴ are independently selected from —F, —Cl, and —Br. In some embodiments, R³ and R⁴ are both —Br. In some embodiments, R³ and R⁴ are both —Br. In some embodiments, R³ and R⁴ are both —Cl. In some embodiments, R³ and R⁴ are both —F.

In some embodiments of the methods described herein is a method of treating depression in a patient in need thereof. In some embodiments, the depression is major depressive disorder, treatment-resistant depression, seasonal affective disorder, psychotic depression, postpartum depression, melancholic depression, atypical depression, or catatonic depression. In some embodiments, the depression is bipolar depression, bipolar treatment-resistant depression, disruptive mood dysregulation disorder, persistent depressive disorder, depressed mood, premenstrual dysphoric disorder, medication-induced depressive disorder, postpartum depression, perimenopausal depression, multi-infarct dementia with depression, presenile dementia with depression, senile dementia with depression, vascular dementia with depressed moods, vascular dementia with depression, or unspecified depressive disorder.

In some embodiments of the methods described herein is a method of treating an anxiety disorder in a patient in need thereof. In some embodiments, the anxiety disorder is obsessive compulsive disorder, post-traumatic stress disorder, or a severe phobia. In some embodiments, the severe phobia is agoraphobia or social phobia.

In some embodiments of the methods described herein is a method of treating pain in a patient in need thereof. In some embodiments, the pain is selected from migraine pain, chronic pain, chronic nerve pain, chronic muscle pain, chronic joint pain, diabetic neuropathy, fibromyalgia, back pain, and osteoarthritis pain.

In some embodiments is a method of treating depression, anxiety disorders, or pain in a patient in need thereof further comprising administering to the patient a peripherally restricted FAAH inhibitor. In some embodiments is a method of treating depression, anxiety disorders, or pain in a patient in need thereof further comprising administering to the patient a peripherally restricted FAAH inhibitor, wherein the peripherally restricted FAAH inhibitor is ASP-3652.

In some embodiments is a method of treating depression, anxiety disorders, or pain in a patient in need thereof further comprising administering to the patient a selective serotonin reuptake inhibitor (SSRI) or a serotonin and norepinephrine reuptake inhibitor (SRNI). In some embodiments is a method of treating depression, anxiety disorders, or pain in a patient in need thereof further comprising administering to the patient a selective serotonin reuptake inhibitor (SSRI). In some embodiments is a method of treating depression, anxiety disorders, or pain in a patient in need thereof further comprising administering to the patient a selective serotonin reuptake inhibitor (SSRI), wherein the selective serotonin reuptake inhibitor (SSRI) is citalopram, escitalopram, fluoxetine, fluvoxamine, paroxetine, or sertraline. In some embodiments is a method of treating depression, anxiety disorders, or pain in a patient in need thereof further comprising administering to the patient a serotonin and norepinephrine reuptake inhibitor (SRNI). In some embodiments is a method of treating depression, anxiety disorders, or pain in a patient in need thereof further comprising administering to the patient a serotonin and norepinephrine reuptake inhibitor (SRNI), wherein the serotonin and norepinephrine reuptake inhibitor (SRNI) is desvenlafaxine, duloxetine, levomilnacipran, milnacipran, sibutramine, tramadol, or venlafaxine.

### BRIEF DESCRIPTION OF THE DRAWINGS

The novel features of the disclosure are set forth with particularity in the appended claims. A better understanding of the features and advantages of the present disclosures will be obtained by reference to the following detailed description that sets forth illustrative embodiments, in which the principles of the disclosures are utilized, and the accompanying drawings of which:

FIG. 1 depicts TRβ target engagement in brain is demonstrated by increased expression of T3-responsive target genes in vivo.

FIG. 2 depicts brain and plasma concentration following 21 days of repeat administration of LL-341070 measured 4 hours post-final dose.

FIG. 3 depicts FAAH expression and specific activity across species and tissue types.

FIG. 4 depicts concentrations of ABX-002A in brain, liver, kidney, lung, and heart were measured 1 hour after SC administration of 30 different prodrugs of ABX-002A.

FIG. 5 depicts plasma, liver, and brain concentrations following ABX-002 prodrug treatment with or without peripheral or global FAAH inhibitors.

FIG. 6A depicts induction of T3-target genes in brain vs. liver after single administration of ABX-002A.

FIG. 6B depicts induction of T3-target genes in brain vs. liver after single administration of ABX-002.

FIG. 6C depicts induction of T3-target genes in brain vs. liver after single administration of ABX-002 plus FAAH inhibitor.

FIG. 7A depicts gene expression in brain and liver, and effects on T4 after administration of ABX-002A.

FIG. 7B depicts gene expression in brain and liver, and effects on T4 after administration of ABX-002.

FIG. 7C depicts gene expression in brain and liver, and effects on T4 after administration of ABX-002 plus peripheral FAAH inhibitor.

FIG. 7D depicts gene expression in brain and liver, and effects on T4 after administration of ABX-002 plus global FAAH inhibitor.

### DETAILED DESCRIPTION OF THE INVENTION

Typical antidepressants (i.e., selective serotonin reupdate inhibitors) are thought to improve major depressive disorder (MDD) through their ability to increase synaptic 5-HT concentrations in brain areas involved in affective tone, including the dorsal raphe, prefrontal cortex, hippocampus, amygdala and hypothalamus. Nonclinical experience using combinations of triiodothyronine (T3) together with antidepressants suggests a mechanism of action involving the enhancement of 5-HT function, including increased 5-HT release and downregulation of inhibitory mechanisms involved in 5-HT release. Specifically, T3 has been shown to augment antidepressant-stimulated 5-HT release in the prefrontal cortex, in part due to enhanced downregulation of inhibitory 5-HT1A and 1B autoreceptors and their signaling within the dorsal raphe. Clinical experience with T3 augmentation in MDD is supported by clinical practice patterns and guidance and from published literature. The largest randomized study to date on treatment alternatives to relieve depression (STAR*D) incorporated T3 treatment as an alternative to augment an inadequate antidepressant response. Specifically, T3 augmentation increased remission rates in patients failing two previous antidepressant regimens. Dose levels of T3 are limited by the activity of the hormone in the peripheral tissue, namely the heart and bone. As shown in Jonklaas et al., (2015), the therapeutically relevant dose of 50 μg both increases heart rate and decreases TSH in the acute setting. Identification of an analog with improved therapeutic index is highly desirable for clinical use in MDD and other disorders. Some approaches to do this focused on enhancing TRb selectivity because the heart and bone are highly enriched for TRa. As an example, the peripherally restricted molecule, resmetirom is highly selective for TRb. However, resmetirom and other thyromimetics are not brain penetrant and thus not able useful for the treatment of MDD.

The compounds disclosed herein are prodrugs of potent thyroid hormone beta receptor-selective agonists that are expected to differentiate from thyroid hormone based on brain-enhanced effects with reduced peripheral side-effects in patients suffering from MDD and with an inadequate response to antidepressants. The compounds are brain-directed thyromimetic prodrugs activated by the intracellular enzyme fatty acid amide hydrolase (FAAH). In particular, ABX-002 is an orally administered amide prodrug exerting its effect as a thyroid hormone receptor full agonist. Upon entry into tissue, FAAH hydrolyzes the amide to release the active carboxylic acid compound, ABX-002A. ABX-002A is a full agonist of both TRb and TRa with 15-fold selectivity for TRβ which helps it avoid effects in both heart and bone. In humans, FAAH is ubiquitously expressed but is enriched in the central nervous system (CNS) resulting in enhanced delivery to the brain and further enrichment in hippocampus and cortex, both regions of the brain implicated in MDD. In mice, oral administration of ABX-002 improves the selective delivery of active metabolite to the brain by >30× compared to dosing active metabolite, resulting in brain-to-plasma ratios >0.8 and brain-to-heart ratios >3.5 In rodent studies, exogenous T3 administration results in higher T3 concentrations in the heart than brain which translates to changes in gene expression at lower doses than in the brain consistent with the dose limiting toxicity described in patients. ABX-002's selective delivery to the CNS allows for doses that maximize the CNS benefit of thyroid hormone agonism without the concomitant dose-limiting adverse effects on peripheral tissues observed with T3 treatment. Selective distribution to the CNS tissue coupled with receptor selectivity for TRβ, gives ABX-002A the potential to offer safety advantages relative to triiodothyronine (T3).

### Certain Terminology

The singular forms “a,” “an,” and “the” include plural referents unless the context clearly dictates otherwise. Thus, for example, reference to “a drug” includes reference to one or more of such drugs, and reference to “an excipient” includes reference to one or more of such excipients.

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range.

The terms “formulation” and “composition,” as used herein, are used interchangeably and refer to a mixture of two or more compounds, elements, or molecules. In some aspects the terms “formulation” and “composition” may be used to refer to a mixture of one or more active agents with a carrier or other excipients.

The terms “active agent,” “active pharmaceutical agent,” “drug,” “active ingredient,” and variants thereof are used interchangeably to refer to an agent or substance that has measurable specified or selected physiologic activity when administered to a subject in a significant or effective amount.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts, and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but not limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol.

The terms “effective amount” or “therapeutically effective amount” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “subject,” “individual,” and “patient” are used interchangeably herein to refer to a mammal. Mammals include, but are not limited to, murines, simians, humans, farm animals, sport animals, and pets.

The term “peripherally restricted FAAH inhibitor” as used herein, refers to a fatty acid amide hydrolase (FAAH) inhibitor that inhibits FAAH to a greater extent in the periphery than in the central nervous system from a systemic dose. In some embodiments, the peripherally restricted FAAH inhibitor is 60% peripherally restricted. In some embodiments, the peripherally restricted FAAH inhibitor is 70% peripherally restricted. In some embodiments, the peripherally restricted FAAH inhibitor is 80% peripherally restricted. In some embodiments, the peripherally restricted FAAH inhibitor is 90% peripherally restricted. In some embodiments, the peripherally restricted FAAH inhibitor is 95% peripherally restricted.

### Target

Thyroid hormone (TH) is a key signal for oligodendrocyte differentiation and myelin formation during development, and also stimulates remyelination in adult models of multiple sclerosis (MS) (Calzá L et al, Brain Res Revs 48:339-346, 2005). However, TH is not an acceptable long-term therapy due to there being virtually no therapeutic window in which remyelination can be achieved while avoiding the cardiotoxicity and bone demineralization associated with chronic hyperthyroidism. Some thyroid hormone analogs can activate thyroid hormone-responsive genes while avoiding the associated downsides of TH by exploiting molecular and physiological features of thyroid hormone receptors (Malm J et al, Mini Rev Med Chem 7:79-86, 2007). These receptors are expressed in two major forms with heterogenous tissue distributions and overlapping but distinct sets of target genes (Yen P M, Physiol Rev 81:1097-1142, 2001). TRa is enriched in the heart, brain, and bone while TRβ is enriched in the liver (O'Shea P J et al, Nucl Recept Signal 4:e011, 2006).

Developing selective thyromimetics has been challenging due to the high sequence homology of thyroid hormone receptor subtypes; namely, only one amino acid residue on the internal surface of the ligand binding domain cavity varies between the α1 and β1 forms.

### Methods

In some embodiments described herein is a method of treating depression, anxiety disorders, or pain in a patient in need thereof comprising administering to the patient a therapeutically effective amount of a compound of Formula (I), or a pharmaceutically acceptable salt or solvate thereof. In some embodiments described herein is a method of treating depression in a patient in need thereof comprising administering to the patient a therapeutically effective amount of a compound of Formula (I), or a pharmaceutically acceptable salt or solvate thereof.

In some embodiments described herein is a method of treating depression in a patient in need thereof comprising administering to the patient a therapeutically effective amount of a compound of Formula (I), or a pharmaceutically acceptable salt or solvate thereof, further comprising administering to the patient a peripherally restricted FAAH inhibitor. In some embodiments described herein is a method of treating depression in a patient in need thereof comprising administering to the patient a therapeutically effective amount of a compound of Formula (I), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating an anxiety disorder in a patient in need thereof comprising administering to the patient a therapeutically effective amount of a compound of Formula (I), or a pharmaceutically acceptable salt or solvate thereof. In some embodiments described herein is a method of treating an anxiety disorder in a patient in need thereof comprising administering to the patient a therapeutically effective amount of a compound of Formula (I), or a pharmaceutically acceptable salt or solvate thereof, further comprising administering to the patient a peripherally restricted FAAH inhibitor. In some embodiments described herein is a method of treating an anxiety disorder in a patient in need thereof comprising administering to the patient a therapeutically effective amount of a compound of Formula (I), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating pain in a patient in need thereof comprising administering to the patient a therapeutically effective amount of a compound of Formula (I), or a pharmaceutically acceptable salt or solvate thereof. In some embodiments described herein is a method of treating pain in a patient in need thereof comprising administering to the patient a therapeutically effective amount of a compound of Formula (I), or a pharmaceutically acceptable salt or solvate thereof, further comprising administering to the patient a peripherally restricted FAAH inhibitor. In some embodiments described herein is a method of treating pain in a patient in need thereof comprising administering to the patient a therapeutically effective amount of a compound of Formula (I), or a pharmaceutically acceptable salt or solvate thereof, further comprising administering to the patient a peripherally restricted FAAH inhibitor, wherein the peripherally restricted FAAH inhibitor is ASP-3652.

In some embodiments described herein is a method of treating depression, anxiety disorders, or pain in a patient in need thereof comprising administering to the patient a therapeutically effective amount of a compound of Formula (II), or a pharmaceutically acceptable salt or solvate thereof. In some embodiments described herein is a method of treating depression in a patient in need thereof comprising administering to the patient a therapeutically effective amount of a compound of Formula (II), or a pharmaceutically acceptable salt or solvate thereof. In some embodiments described herein is a method of treating depression in a patient in need thereof comprising administering to the patient a therapeutically effective amount of a compound of Formula (II), or a pharmaceutically acceptable salt or solvate thereof, further comprising administering to the patient a peripherally restricted FAAH inhibitor. In some embodiments described herein is a method of treating depression in a patient in need thereof comprising administering to the patient a therapeutically effective amount of a compound of Formula (II), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating an anxiety disorder in a patient in need thereof comprising administering to the patient a therapeutically effective amount of a compound of Formula (II), or a pharmaceutically acceptable salt or solvate thereof. In some embodiments described herein is a method of treating an anxiety disorder in a patient in need thereof comprising administering to the patient a therapeutically effective amount of a compound of Formula (II), or a pharmaceutically acceptable salt or solvate thereof, further comprising administering to the patient a peripherally restricted FAAH inhibitor. In some embodiments described herein is a method of treating an anxiety disorder in a patient in need thereof comprising administering to the patient a therapeutically effective amount of a compound of Formula (II), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating pain in a patient in need thereof comprising administering to the patient a therapeutically effective amount of a compound of Formula (II), or a pharmaceutically acceptable salt or solvate thereof. In some embodiments described herein is a method of treating pain in a patient in need thereof comprising administering to the patient a therapeutically effective amount of a compound of Formula (II), or a pharmaceutically acceptable salt or solvate thereof, further comprising administering to the patient a peripherally restricted FAAH inhibitor. In some embodiments described herein is a method of treating pain in a patient in need thereof comprising administering to the patient a therapeutically effective amount of a compound of Formula (II), or a pharmaceutically acceptable salt or solvate thereof, further comprising administering to the patient a peripherally restricted FAAH inhibitor, wherein the peripherally restricted FAAH inhibitor is ASP-3652.

In some embodiments described herein is a method of treating depression, anxiety disorders, or pain in a patient in need thereof comprising administering to the patient a therapeutically effective amount of a compound of Formula (III), or a pharmaceutically acceptable salt or solvate thereof. In some embodiments described herein is a method of treating depression in a patient in need thereof comprising administering to the patient a therapeutically effective amount of a compound of Formula (III), or a pharmaceutically acceptable salt or solvate thereof. In some embodiments described herein is a method of treating depression in a patient in need thereof comprising administering to the patient a therapeutically effective amount of a compound of Formula (III), or a pharmaceutically acceptable salt or solvate thereof, further comprising administering to the patient a peripherally restricted FAAH inhibitor. In some embodiments described herein is a method of treating depression in a patient in need thereof comprising administering to the patient a therapeutically effective amount of a compound of Formula (III), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating an anxiety disorder in a patient in need thereof comprising administering to the patient a therapeutically effective amount of a compound of Formula (III), or a pharmaceutically acceptable salt or solvate thereof. In some embodiments described herein is a method of treating an anxiety disorder in a patient in need thereof comprising administering to the patient a therapeutically effective amount of a compound of Formula (III), or a pharmaceutically acceptable salt or solvate thereof, further comprising administering to the patient a peripherally restricted FAAH inhibitor. In some embodiments described herein is a method of treating an anxiety disorder in a patient in need thereof comprising administering to the patient a therapeutically effective amount of a compound of Formula (III), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating pain in a patient in need thereof comprising administering to the patient a therapeutically effective amount of a compound of Formula (III), or a pharmaceutically acceptable salt or solvate thereof. In some embodiments described herein is a method of treating pain in a patient in need thereof comprising administering to the patient a therapeutically effective amount of a compound of Formula (III), or a pharmaceutically acceptable salt or solvate thereof, further comprising administering to the patient a peripherally restricted FAAH inhibitor. In some embodiments described herein is a method of treating pain in a patient in need thereof comprising administering to the patient a therapeutically effective amount of a compound of Formula (III), or a pharmaceutically acceptable salt or solvate thereof, further comprising administering to the patient a peripherally restricted FAAH inhibitor, wherein the peripherally restricted FAAH inhibitor is ASP-3652.

In some embodiments described herein is a method of treating depression, anxiety disorders, or pain in a patient in need thereof comprising administering to the patient a therapeutically effective amount of a compound of Formula (IV), or a pharmaceutically acceptable salt or solvate thereof. In some embodiments described herein is a method of treating depression in a patient in need thereof comprising administering to the patient a therapeutically effective amount of a compound of Formula (IV), or a pharmaceutically acceptable salt or solvate thereof. In some embodiments described herein is a method of treating depression in a patient in need thereof comprising administering to the patient a therapeutically effective amount of a compound of Formula (IV), or a pharmaceutically acceptable salt or solvate thereof, further comprising administering to the patient a peripherally restricted FAAH inhibitor. In some embodiments described herein is a method of treating depression in a patient in need thereof comprising administering to the patient a therapeutically effective amount of a compound of Formula (IV), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating an anxiety disorder in a patient in need thereof comprising administering to the patient a therapeutically effective amount of a compound of Formula (IV), or a pharmaceutically acceptable salt or solvate thereof. In some embodiments described herein is a method of treating an anxiety disorder in a patient in need thereof comprising administering to the patient a therapeutically effective amount of a compound of Formula (IV), or a pharmaceutically acceptable salt or solvate thereof, further comprising administering to the patient a peripherally restricted FAAH inhibitor. In some embodiments described herein is a method of treating an anxiety disorder in a patient in need thereof comprising administering to the patient a therapeutically effective amount of a compound of Formula (IV), or a pharmaceutically acceptable salt or solvate thereof, further comprising administering to the patient a peripherally restricted FAAH inhibitor, wherein the peripherally restricted FAAH inhibitor is ASP-3652. In some embodiments described herein is a method of treating pain in a patient in need thereof comprising administering to the patient a therapeutically effective amount of a compound of Formula (IV), or a pharmaceutically acceptable salt or solvate thereof. In some embodiments described herein is a method of treating pain in a patient in need thereof comprising administering to the patient a therapeutically effective amount of a compound of Formula (IV), or a pharmaceutically acceptable salt or solvate thereof, further comprising administering to the patient a peripherally restricted FAAH inhibitor. In some embodiments described herein is a method of treating pain in a patient in need thereof comprising administering to the patient a therapeutically effective amount of a compound of Formula (IV), or a pharmaceutically acceptable salt or solvate thereof, further comprising administering to the patient a peripherally restricted FAAH inhibitor, wherein the peripherally restricted FAAH inhibitor is ASP-3652.

In some embodiments of the methods of treating depression described herein, the depression is major depressive disorder, treatment-resistant depression, seasonal affective disorder, psychotic depression, postpartum depression, melancholic depression, atypical depression, or catatonic depression. In some embodiments of the methods of treating depression described herein, the depression is major depressive disorder. In some embodiments of the methods of treating depression described herein, the depression is treatment-resistant depression.

In some embodiments of the methods of treating depression described herein, the depression is seasonal affective disorder. In some embodiments of the methods of treating depression described herein, the depression is psychotic depression. In some embodiments of the methods of treating depression described herein, the depression is postpartum depression. In some embodiments of the methods of treating depression described herein, the depression is melancholic depression. In some embodiments of the methods of treating depression described herein, the depression is atypical depression. In some embodiments of the methods of treating depression described herein, the depression is catatonic depression.

In some embodiments of the methods of treating depression described herein, the depression is bipolar depression, bipolar treatment-resistant depression, disruptive mood dysregulation disorder, persistent depressive disorder, depressed mood, premenstrual dysphoric disorder, medication-induced depressive disorder, postpartum depression, perimenopausal depression, multi-infarct dementia with depression, presenile dementia with depression, senile dementia with depression, vascular dementia with depressed moods, vascular dementia with depression, or unspecified depressive disorder. In some embodiments of the methods of treating depression described herein, the depression is bipolar depression. In some embodiments of the methods of treating depression described herein, the depression is bipolar treatment-resistant depression. In some embodiments of the methods of treating depression described herein, the depression is disruptive mood dysregulation disorder. In some embodiments of the methods of treating depression described herein, the depression is persistent depressive disorder. In some embodiments of the methods of treating depression described herein, the depression is depressed mood. In some embodiments of the methods of treating depression described herein, the depression is premenstrual dysphoric disorder. In some embodiments of the methods of treating depression described herein, the depression is medication-induced depressive disorder. In some embodiments of the methods of treating depression described herein, the depression is postpartum depression. In some embodiments of the methods of treating depression described herein, the depression is perimenopausal depression. In some embodiments of the methods of treating depression described herein, the depression is multi-infarct dementia with depression.

In some embodiments of the methods of treating depression described herein, the depression is presenile dementia with depression. In some embodiments of the methods of treating depression described herein, the depression is senile dementia with depression. In some embodiments of the methods of treating depression described herein, the depression is vascular dementia with depressed moods. In some embodiments of the methods of treating depression described herein, the depression is vascular dementia with depression. In some embodiments of the methods of treating depression described herein, the depression is unspecified depressive disorder.

In some embodiments of the methods of treating an anxiety disorder described herein, the anxiety disorder is obsessive compulsive disorder, post-traumatic stress disorder, or a severe phobia. In some embodiments of the methods of treating an anxiety disorder described herein, the anxiety disorder is obsessive compulsive disorder. In some embodiments of the methods of treating an anxiety disorder described herein, the anxiety disorder is post-traumatic stress disorder. In some embodiments of the methods of treating an anxiety disorder described herein, the anxiety disorder is a severe phobia. In some embodiments of the methods of treating an anxiety disorder described herein, the anxiety disorder is a severe phobia, wherein the severe phobia is agoraphobia or social phobia. In some embodiments of the methods of treating an anxiety disorder described herein, the anxiety disorder is a severe phobia, wherein the severe phobia is agoraphobia. In some embodiments of the methods of treating an anxiety disorder described herein, the anxiety disorder is a severe phobia, wherein the severe phobia is social phobia.

In some embodiments of the methods of treating pain described herein, the pain is selected from migraine pain, chronic pain, chronic nerve pain, chronic muscle pain, chronic joint pain, diabetic neuropathy, fibromyalgia, back pain, and osteoarthritis pain. In some embodiments of the methods of treating pain described herein, the pain is migraine pain. In some embodiments of the methods of treating pain described herein, the pain is chronic pain. In some embodiments of the methods of treating pain described herein, the pain is chronic nerve pain. In some embodiments of the methods of treating pain described herein, the pain is chronic muscle pain. In some embodiments of the methods of treating pain described herein, the pain is chronic joint pain. In some embodiments of the methods of treating pain described herein, the pain is diabetic neuropathy. In some embodiments of the methods of treating pain described herein, the pain is fibromyalgia. In some embodiments of the methods of treating pain described herein, the pain is back pain. In some embodiments of the methods of treating pain described herein, the pain is osteoarthritis pain.

In some embodiments, the methods of treatment described herein further comprise administering to the patient a selective serotonin reuptake inhibitor (SSRI) or a serotonin and norepinephrine reuptake inhibitor (SRNI).

In some embodiments, the methods of treatment described herein further comprise administering to the patient a selective serotonin reuptake inhibitor (SSRI). In some embodiments, the methods of treatment described herein further comprise administering to the patient a selective serotonin reuptake inhibitor (SSRI), wherein the SSRI is citalopram, escitalopram, fluoxetine, fluvoxamine, paroxetine, or sertraline. In some embodiments, the methods of treatment described herein further comprise administering to the patient a selective serotonin reuptake inhibitor (SSRI), wherein the SSRI is citalopram. In some embodiments, the methods of treatment described herein further comprise administering to the patient a selective serotonin reuptake inhibitor (SSRI), wherein the SSRI is escitalopram. In some embodiments, the methods of treatment described herein further comprise administering to the patient a selective serotonin reuptake inhibitor (SSRI), wherein the SSRI is fluoxetine. In some embodiments, the methods of treatment described herein further comprise administering to the patient a selective serotonin reuptake inhibitor (SSRI), wherein the SSRI is fluvoxamine. In some embodiments, the methods of treatment described herein further comprise administering to the patient a selective serotonin reuptake inhibitor (SSRI), wherein the SSRI is paroxetine. In some embodiments, the methods of treatment described herein further comprise administering to the patient a selective serotonin reuptake inhibitor (SSRI), wherein the SSRI is sertraline.

In some embodiments, the methods of treatment described herein further comprise administering to the patient serotonin and norepinephrine reuptake inhibitor (SRNI), wherein the SRNI is desvenlafaxine, duloxetine, levomilnacipran, milnacipran, sibutramine, tramadol, or venlafaxine. In some embodiments, the methods of treatment described herein further comprise administering to the patient serotonin and norepinephrine reuptake inhibitor (SRNI), wherein the SRNI is desvenlafaxine. In some embodiments, the methods of treatment described herein further comprise administering to the patient serotonin and norepinephrine reuptake inhibitor (SRNI), wherein the SRNI is duloxetine. In some embodiments, the methods of treatment described herein further comprise administering to the patient serotonin and norepinephrine reuptake inhibitor (SRNI), wherein the SRNI is levomilnacipran. In some embodiments, the methods of treatment described herein further comprise administering to the patient serotonin and norepinephrine reuptake inhibitor (SRNI), wherein the SRNI is milnacipran. In some embodiments, the methods of treatment described herein further comprise administering to the patient serotonin and norepinephrine reuptake inhibitor (SRNI), wherein the SRNI is sibutramine. In some embodiments, the methods of treatment described herein further comprise administering to the patient serotonin and norepinephrine reuptake inhibitor (SRNI), wherein the SRNI is tramadol. In some embodiments, the methods of treatment described herein further comprise administering to the patient serotonin and norepinephrine reuptake inhibitor (SRNI), wherein the SRNI is venlafaxine.

### Compounds

The compounds described herein are fatty acid amide hydrolase (FAAH) cleavable prodrugs. In some embodiments, the compounds described herein comprise a fatty acid amide hydrolase (FAAH) cleavable prodrug of Formula (I), wherein the prodrug of Formula (I) is a prodrug of a TRβ agonist. In some embodiments, the compounds described herein comprise a fatty acid amide hydrolase (FAAH) cleavable prodrug of Formula (II), wherein the prodrug of Formula (II) is a prodrug of a TRβ agonist. In some embodiments, the compounds described herein comprise a fatty acid amide hydrolase (FAAH) cleavable prodrug of Formula (III), wherein the prodrug of Formula (III) is a prodrug of a TRβ agonist. In some embodiments, the compounds described herein comprise a fatty acid amide hydrolase (FAAH) cleavable prodrug of Formula (IV), wherein the prodrug of Formula (IV) is a prodrug of a TRβ agonist.

In some embodiments described herein is a method of treating depression, anxiety disorders, or pain in a patient in need thereof comprising administering to the patient a therapeutically effective amount of a compound of Formula (I), or a pharmaceutically acceptable salt or solvate thereof:

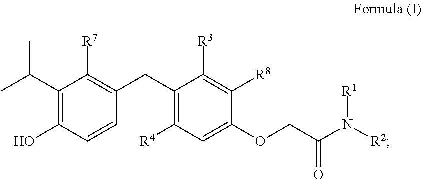

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I;
R⁵ and R⁶ are independently selected from hydrogen and C₁-C₆alkyl; and
R⁷ and R⁸ are independently selected from hydrogen, —F, —Cl, —Br, and —I.

In some embodiments, R⁷ is hydrogen. In some embodiments, R⁷ is —F. In some embodiments, R⁷ is —Cl. In some embodiments, R⁷ is —Br.

In some embodiments, R⁸ is hydrogen. In some embodiments, R⁸ is —F. In some embodiments, R⁸ is —Cl. In some embodiments, R⁸ is —Br.

In some embodiments, R¹ is hydrogen. In some embodiments, R¹ is C₁₋₆alkyl.

In some embodiments, R² is C₁-C₆alkyl optionally substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo. In some embodiments, R² is C₁-C₆alkyl substituted with one cyano. In some embodiments, R² is C₁-C₆alkyl substituted with one or more —OR⁵. In some embodiments, R² is C₁-C₆alkyl substituted with one or more —OH. In some embodiments, R² is C₁-C₆alkyl substituted with one —OH. In some embodiments, R² is C₁-C₆alkyl substituted with one or more —NR⁵R⁶. In some embodiments, R² is C₁-C₆alkyl substituted with one or more —NH₂. In some embodiments, R² is C₁-C₆alkyl substituted with one —NH₂. In some embodiments, R² is C₁-C₆alkyl substituted with one —S(O)₂R⁵. In some embodiments, R² is C₁-C₆alkyl substituted with one —S(O)₂H. In some embodiments, R² is C₁-C₆alkyl substituted with one —S(O)₂OR⁵. In some embodiments, R² is C₁-C₆alkyl substituted with one —S(O)₂OH. In some embodiments, R² is unsubstituted C₁-C₆alkyl. In some embodiments, R² is —CH₃. In some embodiments, R² is —CH₂CH₃. In some embodiments, R² is —CH₂CH₂CH₃.

In some embodiments, R² is C₂-C₆alkenyl optionally substituted with one or more of halo, cyano, —OR⁵, —NR⁵R⁶, —S(O)₂R⁵, or —S(O)₂OR⁵. In some embodiments, R² is unsubstituted C₂-C₆alkenyl.

In some embodiments, R² is C₂-C₆alkynyl optionally substituted with one or more of halo, cyano, —OR⁵, —NR⁵R⁶, —S(O)₂R⁵, or —S(O)₂OR⁵. In some embodiments, R² is unsubstituted C₂-C₆alkynyl.

In some embodiments, R² is C₃-C₆cycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆cycloalkyl.

In some embodiments, R² is C₃-C₆heterocycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆heterocycloalkyl.

In some embodiments, R² is phenyl optionally substituted with one or more of halo, cyano, —OR⁵, —NR⁵R⁶, —S(O)₂R⁵, or —S(O)₂OR⁵. In some embodiments, R² is phenyl substituted with one or more of halo, cyano, —OR⁵, —NR⁵R⁶, —S(O)₂R⁵, or —S(O)₂OR⁵. In some embodiments, R² is phenyl substituted with one or more of halo. In some embodiments, R² is phenyl substituted with one or more —OR⁵. In some embodiments, R² is phenyl substituted with one or more —OH. In some embodiments, R² is unsubstituted phenyl.

In some embodiments, R² is —C₁-C₆alkyl-phenyl optionally substituted with one or more of halo, cyano, —OR⁵, —NR⁵R⁶, —S(O)₂R⁵, or —S(O)₂OR⁵. In some embodiments, R² is unsubstituted —C₁-C₆alkyl-phenyl.

In some embodiments, R² is —OR⁵. In some embodiments, R² is —OH. In some embodiments, R² is —NR⁵R⁶. In some embodiments, R² is —NH₂.

In some embodiments, R² is hydrogen.

In some embodiments, R³ and R⁴ are independently selected from —F, —Cl, and —Br. In some embodiments, R³ and R⁴ are both —Br. In some embodiments, R³ and R⁴ are both —Br. In some embodiments, R³ and R⁴ are both —Cl. In some embodiments, R³ and R⁴ are both —F. In some embodiments, R³ is —Cl and R⁴ is —Br. In some embodiments, R³ is —F and R⁴ is —Br. In some embodiments, R³ is —F and R⁴ is —Cl.

In some embodiments described herein is a method of treating depression, anxiety disorders, or pain in a patient in need thereof comprising administering to the patient a therapeutically effective amount of a compound of Formula (II), or a pharmaceutically acceptable salt or solvate thereof:

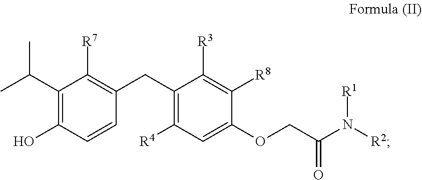

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I;
R⁵ and R⁶ are independently selected from hydrogen and C₁-C₆alkyl; and
R⁷ and R⁸ are independently selected from hydrogen, —F, —Cl, —Br, and —I, wherein at least one of R⁷ and R⁸ is not hydrogen.

In some embodiments, R⁷ is hydrogen. In some embodiments, R⁷ is —F. In some embodiments, R⁷ is —Cl. In some embodiments, R⁷ is —Br.

In some embodiments, R⁷ is hydrogen. In some embodiments, R⁷ is —F. In some embodiments, R⁸ is —Cl. In some embodiments, R⁸ is —Br.

In some embodiments, R¹ is hydrogen. In some embodiments, R¹ is C₁₋₆alkyl.

In some embodiments, R² is C₁-C₆alkyl optionally substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo. In some embodiments, R² is C₁-C₆alkyl substituted with one cyano. In some embodiments, R² is C₁-C₆alkyl substituted with one or more —OR⁵. In some embodiments, R² is C₁-C₆alkyl substituted with one or more —OH. In some embodiments, R² is C₁-C₆alkyl substituted with one —OH. In some embodiments, R² is C₁-C₆alkyl substituted with one or more —NR⁵R⁶. In some embodiments, R² is C₁-C₆alkyl substituted with one or more —NH₂. In some embodiments, R² is C₁-C₆alkyl substituted with one —NH₂. In some embodiments, R² is C₁-C₆alkyl substituted with one —S(O)₂R⁵. In some embodiments, R² is C₁-C₆alkyl substituted with one —S(O)₂H. In some embodiments, R² is C₁-C₆alkyl substituted with one —S(O)₂OR⁵. In some embodiments, R² is C₁-C₆alkyl substituted with one —S(O)₂OH. In some embodiments, R² is unsubstituted C₁-C₆alkyl. In some embodiments, R² is —CH₃. In some embodiments, R² is —CH₂CH₃. In some embodiments, R² is —CH₂CH₂CH₃.

In some embodiments, R² is C₂-C₆alkenyl optionally substituted with one or more of halo, cyano, —OR⁵, —NR⁵R⁶, —S(O)₂R⁵, or —S(O)₂OR⁵. In some embodiments, R² is unsubstituted C₂-C₆alkenyl.

In some embodiments, R² is C₂-C₆alkynyl optionally substituted with one or more of halo, cyano, —OR⁵, —NR⁵R⁶, —S(O)₂R⁵, or —S(O)₂OR⁵. In some embodiments, R² is unsubstituted C₂-C₆alkynyl.

In some embodiments, R² is C₃-C₆cycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆cycloalkyl.

In some embodiments, R² is C₃-C₆heterocycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆heterocycloalkyl.

In some embodiments, R² is phenyl optionally substituted with one or more of halo, cyano, —OR⁵, —NR⁵R⁶, —S(O)₂R⁵, or —S(O)₂OR⁵. In some embodiments, R² is phenyl substituted with one or more of halo, cyano, —OR⁵, —NR⁵R⁶, —S(O)₂R⁵, or —S(O)₂OR⁵. In some embodiments, R² is phenyl substituted with one or more of halo. In some embodiments, R² is phenyl substituted with one or more —OR⁵. In some embodiments, R² is phenyl substituted with one or more —OH. In some embodiments, R² is unsubstituted phenyl.

In some embodiments, R² is —C₁-C₆alkyl-phenyl optionally substituted with one or more of halo, cyano, —OR⁵, —NR⁵R⁶, —S(O)₂R⁵, or —S(O)₂OR⁵. In some embodiments, R² is unsubstituted —C₁-C₆alkyl-phenyl.

In some embodiments, R² is —OR⁵. In some embodiments, R² is —OH. In some embodiments, R² is —NR⁵R⁶. In some embodiments, R² is —NH₂.

In some embodiments, R² is hydrogen.

In some embodiments, R³ and R⁴ are independently selected from —F, —Cl, and —Br. In some embodiments, R³ and R⁴ are both —Br. In some embodiments, R³ and R⁴ are both —Br. In some embodiments, R³ and R⁴ are both —Cl. In some embodiments, R³ and R⁴ are both —F. In some embodiments, R³ is —Cl and R⁴ is —Br. In some embodiments, R³ is —F and R⁴ is —Br. In some embodiments, R³ is —F and R⁴ is —Cl.

In some embodiments described herein is a method of treating depression, anxiety disorders, or pain in a patient in need thereof comprising administering to the patient a therapeutically effective amount of a compound of Formula (III), or a pharmaceutically acceptable salt or solvate thereof:

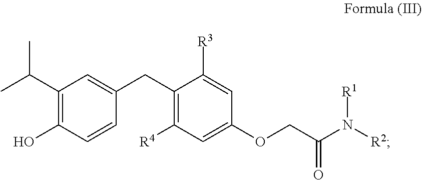

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I; and
R⁵ and R⁶ are independently selected from hydrogen and C₁-C₆alkyl.

In some embodiments, R¹ is hydrogen. In some embodiments, R¹ is C₁₋₆alkyl.

In some embodiments, R² is C₁-C₆alkyl optionally substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo. In some embodiments, R² is C₁-C₆alkyl substituted with one cyano. In some embodiments, R² is C₁-C₆alkyl substituted with one or more —OR⁵. In some embodiments, R² is C₁-C₆alkyl substituted with one or more —OH. In some embodiments, R² is C₁-C₆alkyl substituted with one —OH. In some embodiments, R² is C₁-C₆alkyl substituted with one or more —NR⁵R⁶. In some embodiments, R² is C₁-C₆alkyl substituted with one or more —NH₂. In some embodiments, R² is C₁-C₆alkyl substituted with one —NH₂. In some embodiments, R² is C₁-C₆alkyl substituted with one —S(O)₂R⁵. In some embodiments, R² is C₁-C₆alkyl substituted with one —S(O)₂H. In some embodiments, R² is C₁-C₆alkyl substituted with one —S(O)₂OR⁵. In some embodiments, R² is C₁-C₆alkyl substituted with one —S(O)₂OH. In some embodiments, R² is unsubstituted C₁-C₆alkyl. In some embodiments, R² is —CH₃. In some embodiments, R² is —CH₂CH₃. In some embodiments, R² is —CH₂CH₂CH₃.

In some embodiments, R² is C₂-C₆alkenyl optionally substituted with one or more of halo, cyano, —OR⁵, —NR⁵R⁶, —S(O)₂R⁵, or —S(O)₂OR⁵. In some embodiments, R² is unsubstituted C₂-C₆alkenyl.

In some embodiments, R² is C₂-C₆alkynyl optionally substituted with one or more of halo, cyano, —OR⁵, —NR⁵R⁶, —S(O)₂R⁵, or —S(O)₂OR⁵. In some embodiments, R² is unsubstituted C₂-C₆alkynyl.

In some embodiments, R² is C₃-C₆cycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆cycloalkyl.

In some embodiments, R² is C₃-C₆heterocycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆heterocycloalkyl.

In some embodiments, R² is phenyl optionally substituted with one or more of halo, cyano, —OR⁵, —NR⁵R⁶, —S(O)₂R⁵, or —S(O)₂OR⁵. In some embodiments, R² is phenyl substituted with one or more of halo, cyano, —OR⁵, —NR⁵R⁶, —S(O)₂R⁵, or —S(O)₂OR⁵. In some embodiments, R² is phenyl substituted with one or more of halo. In some embodiments, R² is phenyl substituted with one or more —OR⁵. In some embodiments, R² is phenyl substituted with one or more —OH. In some embodiments, R² is unsubstituted phenyl.

In some embodiments, R² is —C₁-C₆alkyl-phenyl optionally substituted with one or more of halo, cyano, —OR⁵, —NR⁵R⁶, —S(O)₂R⁵, or —S(O)₂OR⁵. In some embodiments, R² is unsubstituted —C₁-C₆alkyl-phenyl.

In some embodiments, R² is —OR⁵. In some embodiments, R² is —OH. In some embodiments, R² is —NR⁵R⁶. In some embodiments, R² is —NH₂.

In some embodiments, R² is hydrogen.

In some embodiments, R³ and R⁴ are independently selected from —F, —Cl, and —Br. In some embodiments, R³ and R⁴ are both —Br. In some embodiments, R³ and R⁴ are both —Br. In some embodiments, R³ and R⁴ are both —Cl. In some embodiments, R³ and R⁴ are both —F. In some embodiments, R³ is —Cl and R⁴ is —Br. In some embodiments, R³ is —F and R⁴ is —Br. In some embodiments, R³ is —F and R⁴ is —Cl.

In some embodiments described herein is a method of treating depression, anxiety disorders, or pain in a patient in need thereof comprising administering to the patient a therapeutically effective amount of a compound of Formula (IV), or a pharmaceutically acceptable salt or solvate thereof:

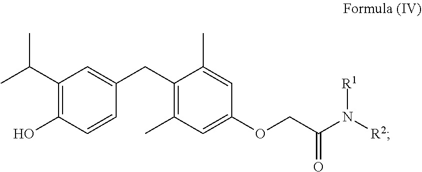

wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵; and
R⁵ and R⁶ are independently selected from hydrogen and C₁-C₆alkyl.

In some embodiments, R¹ is hydrogen. In some embodiments, R¹ is C₁₋₆alkyl.

In some embodiments, R² is C₁-C₆alkyl optionally substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo, cyano, —OR⁵, —NR⁵R⁶, —S(O)₂R⁵, or —S(O)₂OR⁵. In some embodiments, R² is C₁-C₆alkyl substituted with one or more of halo. In some embodiments, R² is C₁-C₆alkyl substituted with one cyano. In some embodiments, R² is C₁-C₆alkyl substituted with one or more —OR⁵. In some embodiments, R² is C₁-C₆alkyl substituted with one or more —OH. In some embodiments, R² is C₁-C₆alkyl substituted with one —OH. In some embodiments, R² is C₁-C₆alkyl substituted with one or more —NR⁵R⁶. In some embodiments, R² is C₁-C₆alkyl substituted with one or more —NH₂. In some embodiments, R² is C₁-C₆alkyl substituted with one —NH₂. In some embodiments, R² is C₁-C₆alkyl substituted with one —S(O)₂R⁵. In some embodiments, R² is C₁-C₆alkyl substituted with one —S(O)₂H. In some embodiments, R² is C₁-C₆alkyl substituted with one —S(O)₂OR⁵. In some embodiments, R² is C₁-C₆alkyl substituted with one —S(O)₂OH. In some embodiments, R² is unsubstituted C₁-C₆alkyl. In some embodiments, R² is —CH₃. In some embodiments, R² is —CH₂CH₃. In some embodiments, R² is —CH₂CH₂CH₃.

In some embodiments, R² is C₂-C₆alkenyl optionally substituted with one or more of halo, cyano, —OR⁵, —NR⁵R⁶, —S(O)₂R⁵, or —S(O)₂OR⁵. In some embodiments, R² is unsubstituted C₂-C₆alkenyl.

In some embodiments, R² is C₂-C₆alkynyl optionally substituted with one or more of halo, cyano, —OR⁵, —NR⁵R⁶, —S(O)₂R⁵, or —S(O)₂OR⁵. In some embodiments, R² is unsubstituted C₂-C₆alkynyl.

In some embodiments, R² is C₃-C₆cycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆cycloalkyl.

In some embodiments, R² is C₃-C₆heterocycloalkyl optionally substituted with one or more of halo, cyano, —OR⁵, —NR⁵R⁶, —S(O)₂R⁵, or —S(O)₂OR⁵. In some embodiments, R² is unsubstituted C₃-C₆heterocycloalkyl.

In some embodiments, R² is phenyl optionally substituted with one or more of halo, cyano, —OR⁵, —NR⁵R⁶, —S(O)₂R⁵, or —S(O)₂OR⁵. In some embodiments, R² is phenyl substituted with one or more of halo, cyano, —OR⁵, —NR⁵R⁶, —S(O)₂R⁵, or —S(O)₂OR⁵. In some embodiments, R² is phenyl substituted with one or more of halo. In some embodiments, R² is phenyl substituted with one or more —OR⁵. In some embodiments, R² is phenyl substituted with one or more —OH. In some embodiments, R² is unsubstituted phenyl.

In some embodiments, R² is —C₁-C₆alkyl-phenyl optionally substituted with one or more of halo, cyano, —OR⁵, —NR⁵R⁶, —S(O)₂R⁵, or —S(O)₂OR⁵. In some embodiments, R² is unsubstituted —C₁-C₆alkyl-phenyl.

In some embodiments, R² is —OR⁵. In some embodiments, R² is —OH. In some embodiments, R² is —NR⁵R⁶. In some embodiments, R² is —NH₂.

In some embodiments, R² is hydrogen.

In some embodiments of the fatty acid amide hydrolase (FAAH) cleavable prodrugs described herein the prodrug has a structure selected from

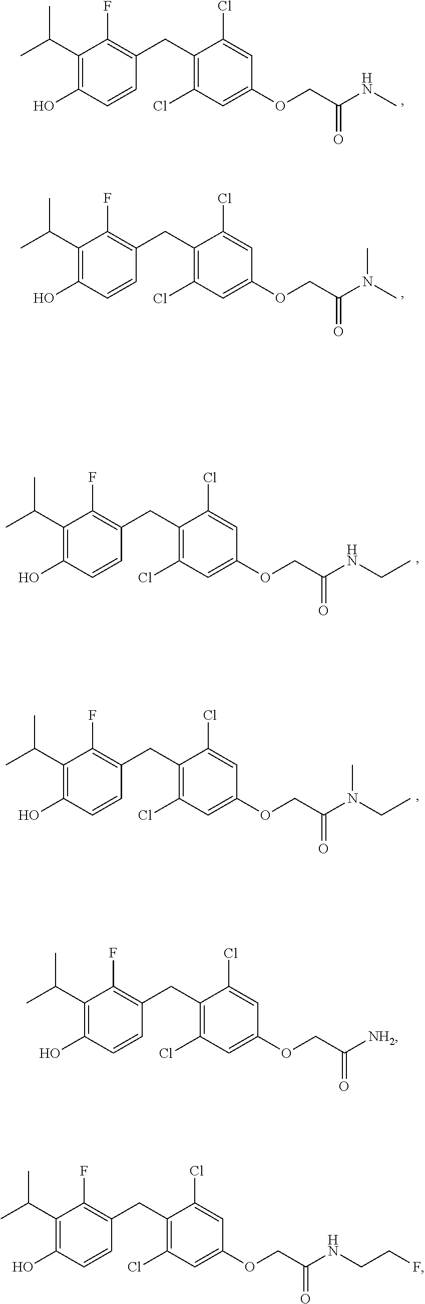

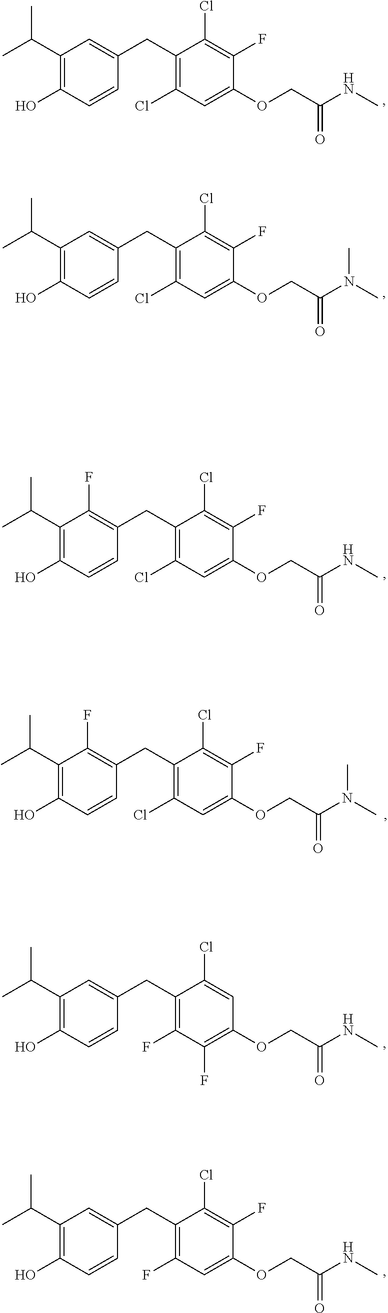

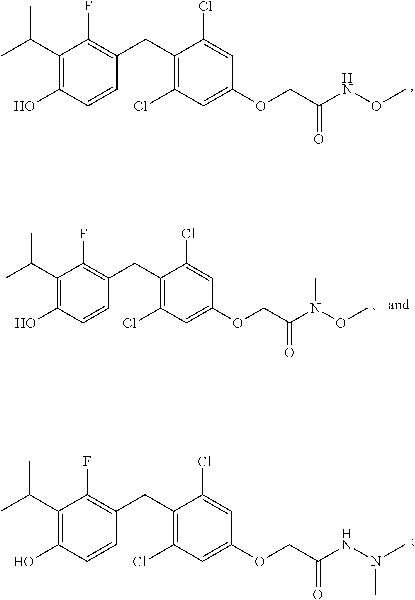

or a pharmaceutically acceptable salt or solvate thereof.

In some embodiments of the fatty acid amide hydrolase (FAAH) cleavable prodrugs described herein, the prodrug has a structure selected from:

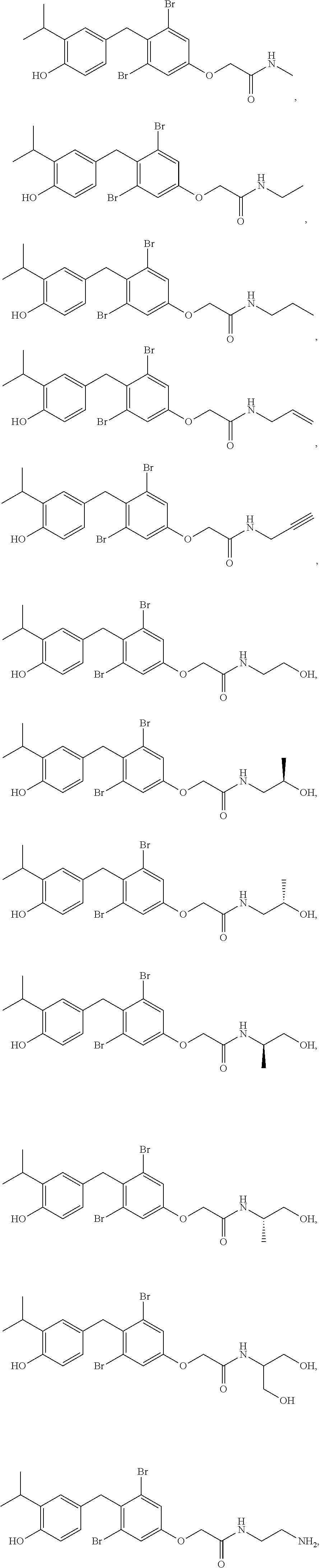

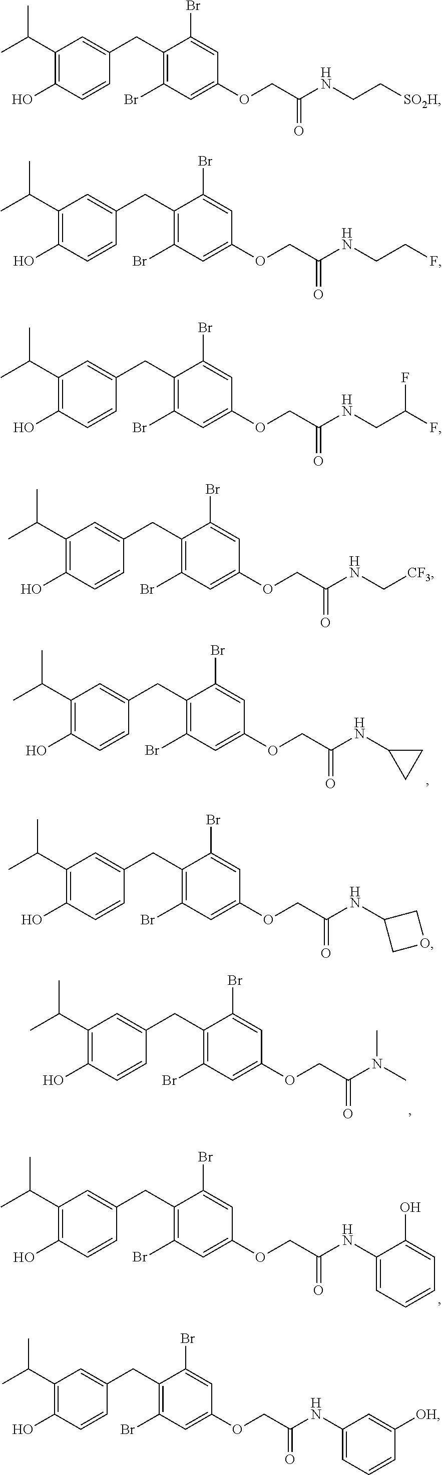

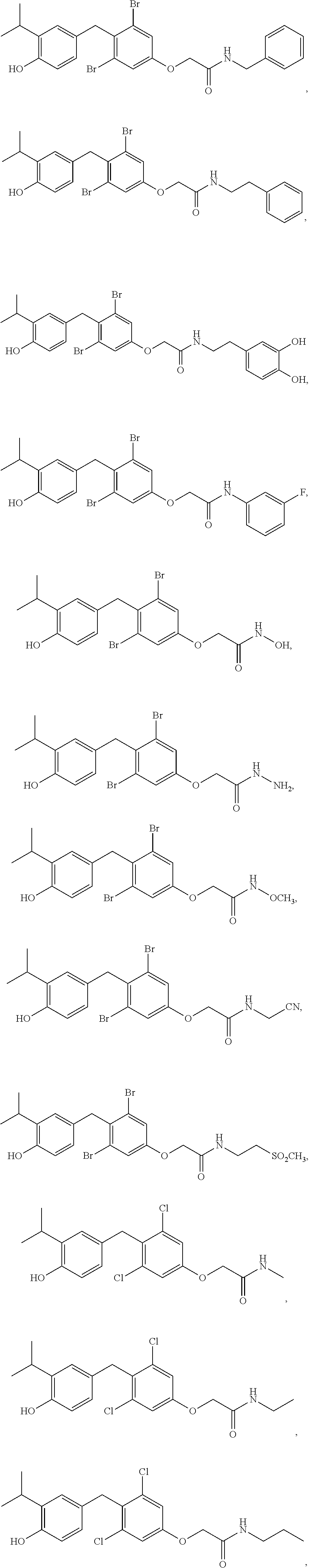

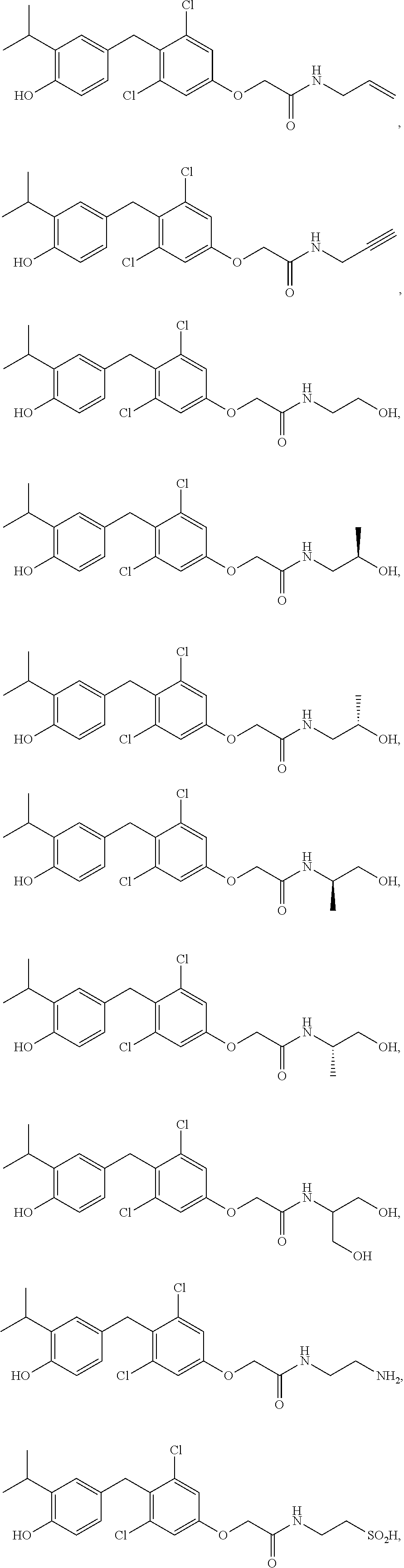

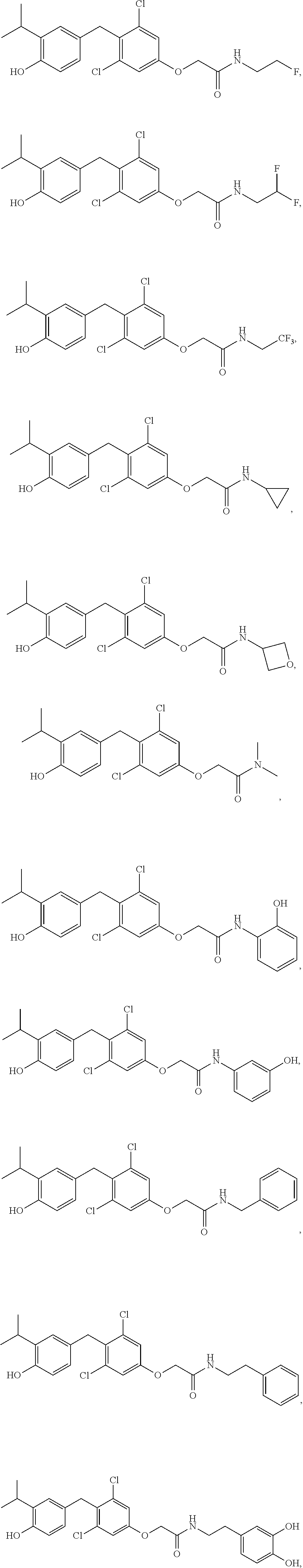

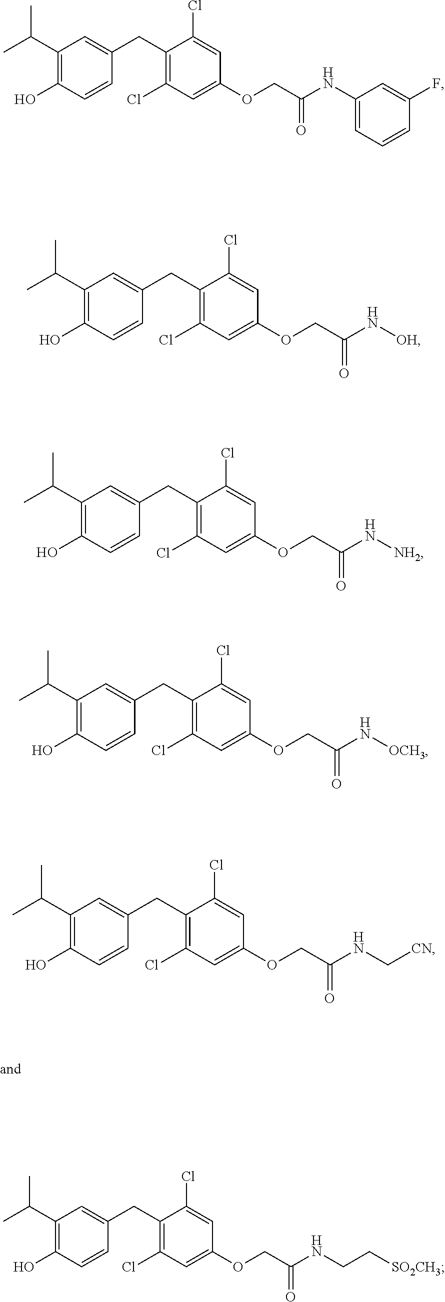 [SMILES: CC(C)c1cc(Cc2c(Cl)cc(OCC(=O)Nc3cccc(F)c3)cc2Cl)ccc1O ; CC(C)c1cc(Cc2c(Cl)cc(OCC(=O)NO)cc2Cl)ccc1O ; CC(C)c1cc(Cc2c(Cl)cc(OCC(=O)NN)cc2Cl)ccc1O ; CONC(=O)COc1cc(Cl)c(Cc2ccc(O)c(C(C)C)c2)c(Cl)c1 ; CC(C)c1cc(Cc2c(Cl)cc(OCC(=O)NCC#N)cc2Cl)ccc1O ; CC(C)c1cc(Cc2c(Cl)cc(OCC(=O)NCCS(C)(=O)=O)cc2Cl)ccc1O]

or a pharmaceutically acceptable salt or solvate thereof.

In some embodiments of the fatty acid amide hydrolase (FAAH) cleavable prodrugs described herein, the prodrug has a structure selected from:

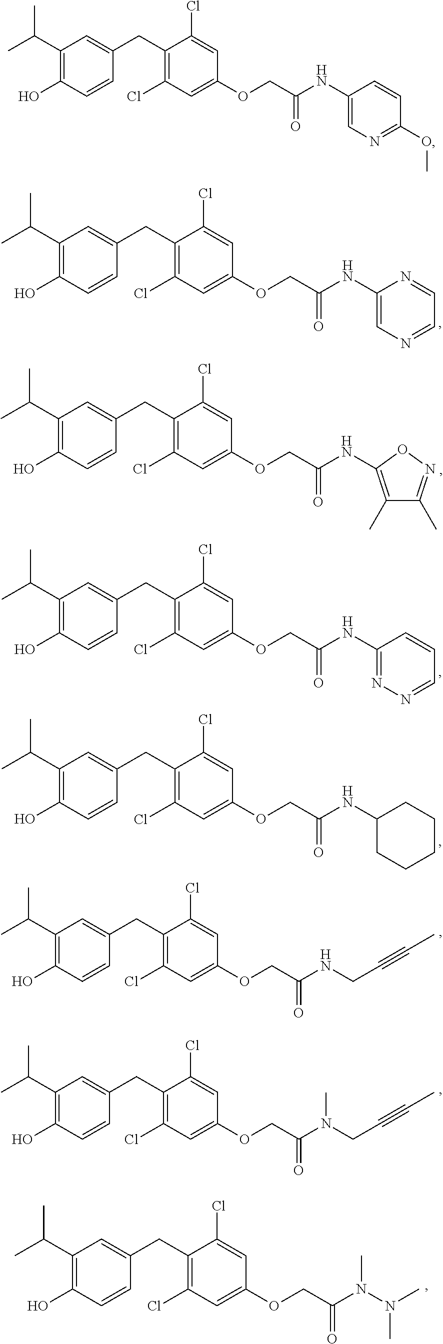

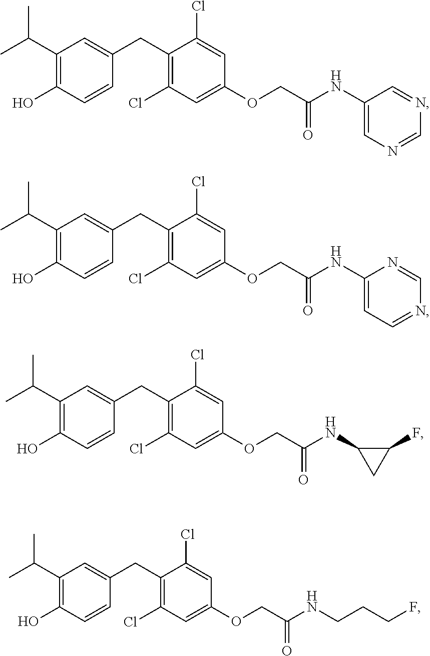 [SMILES: CC(C)c1cc(Cc2c(Cl)cc(OCC(=O)Nc3cncnc3)cc2Cl)ccc1O ; CC(C)c1cc(Cc2c(Cl)cc(OCC(=O)Nc3ccncn3)cc2Cl)ccc1O ; CC(C)c1cc(Cc2c(Cl)cc(OCC(=O)N[C@@H]3C[C@@H]3F)cc2Cl)ccc1O ; CC(C)c1cc(Cc2c(Cl)cc(OCC(=O)NCCCF)cc2Cl)ccc1O]

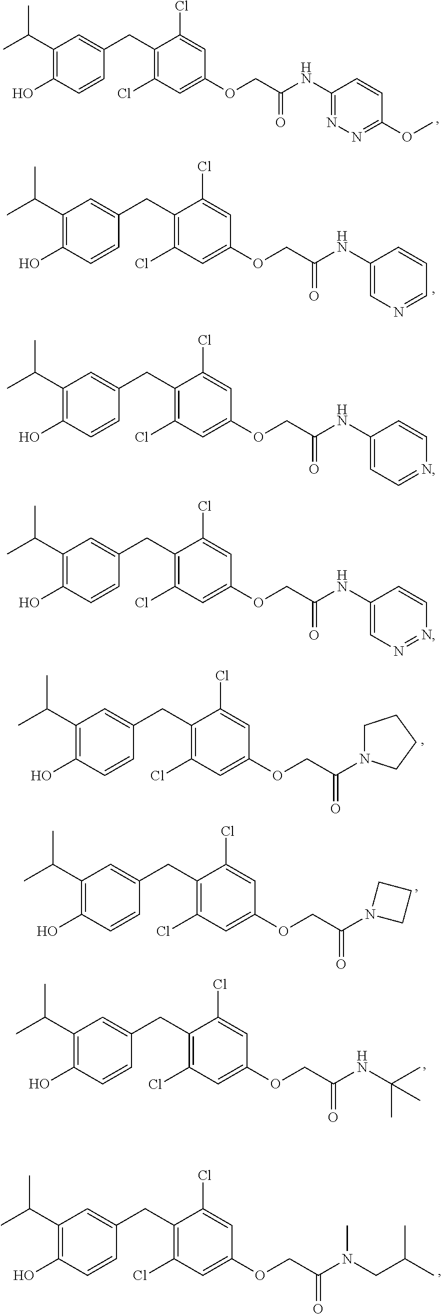

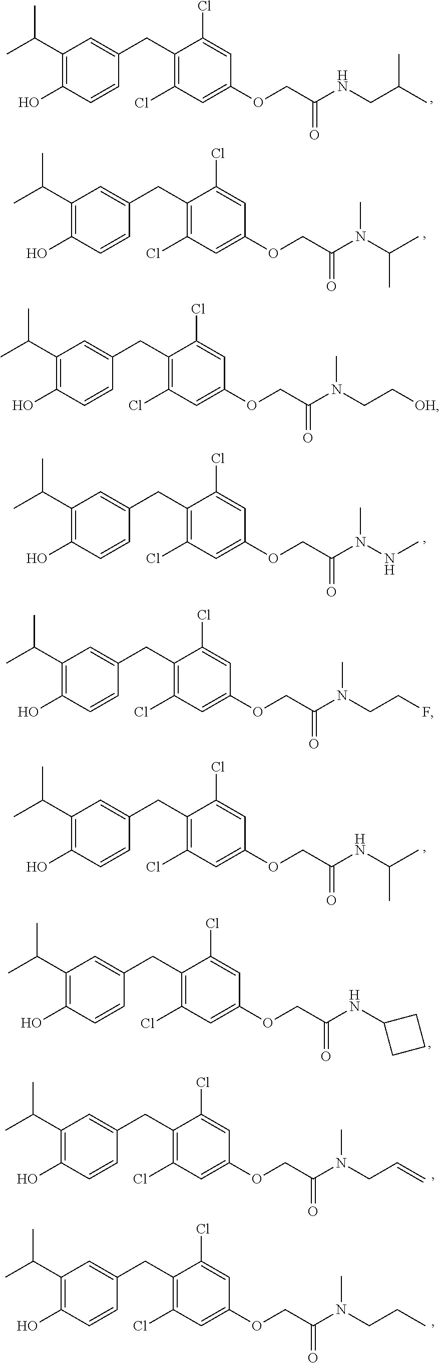

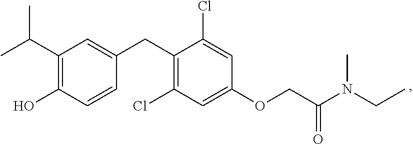 [SMILES: CCN(C)C(=O)COc1cc(Cl)c(Cc2ccc(O)c(C(C)C)c2)c(Cl)c1]

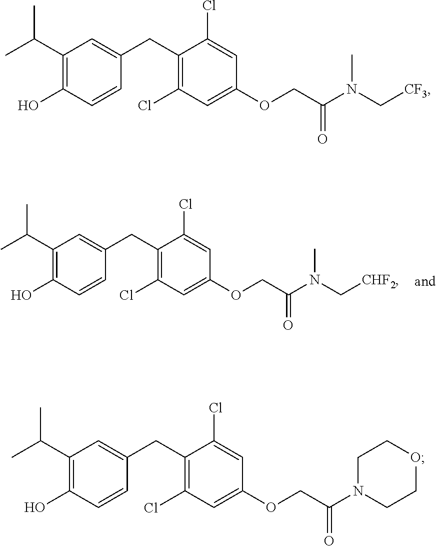 [SMILES: CC(C)c1cc(Cc2c(Cl)cc(OCC(=O)N(C)CC(F)(F)F)cc2Cl)ccc1O ; CC(C)c1cc(Cc2c(Cl)cc(OCC(=O)N(C)CC(F)F)cc2Cl)ccc1O ; CC(C)c1cc(Cc2c(Cl)cc(OCC(=O)N3CCOCC3)cc2Cl)ccc1O]

or a pharmaceutically acceptable salt or solvate thereof.

In some embodiments of the fatty acid amide hydrolase (FAAH) cleavable prodrugs described herein, the prodrug has a structure selected from:

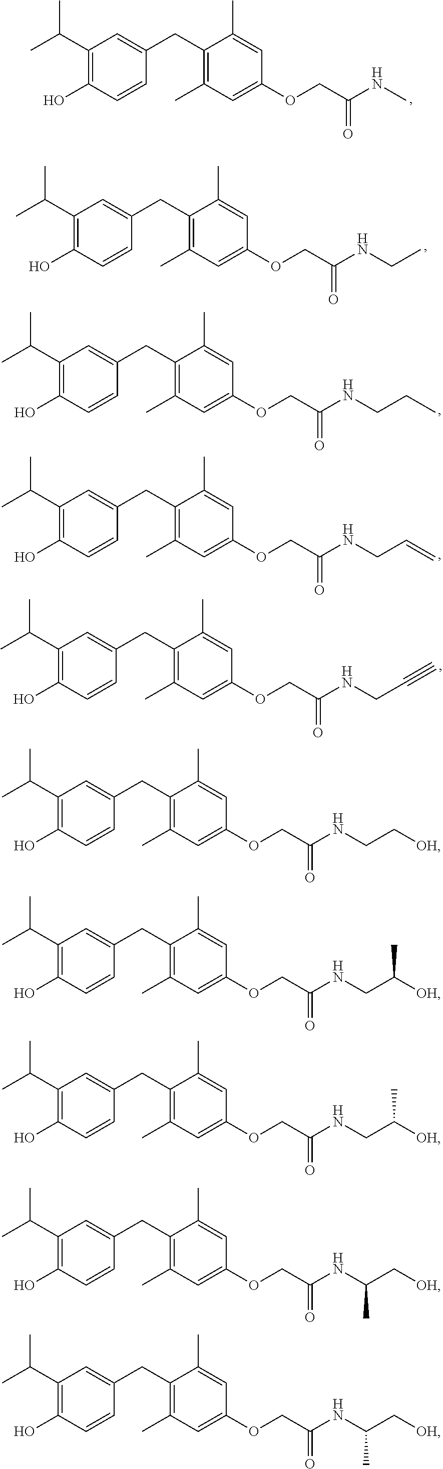

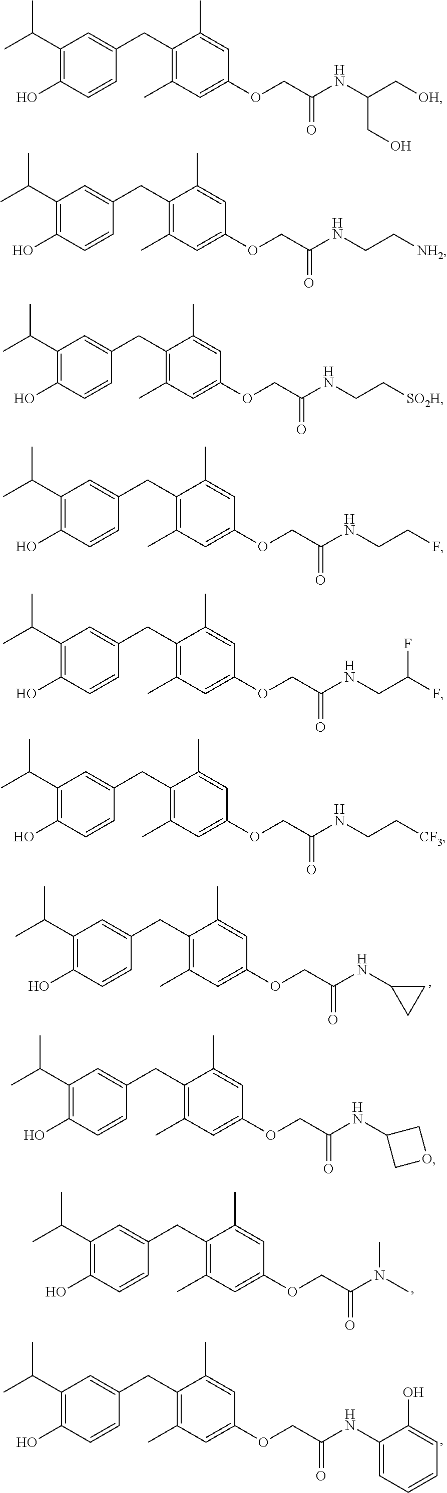

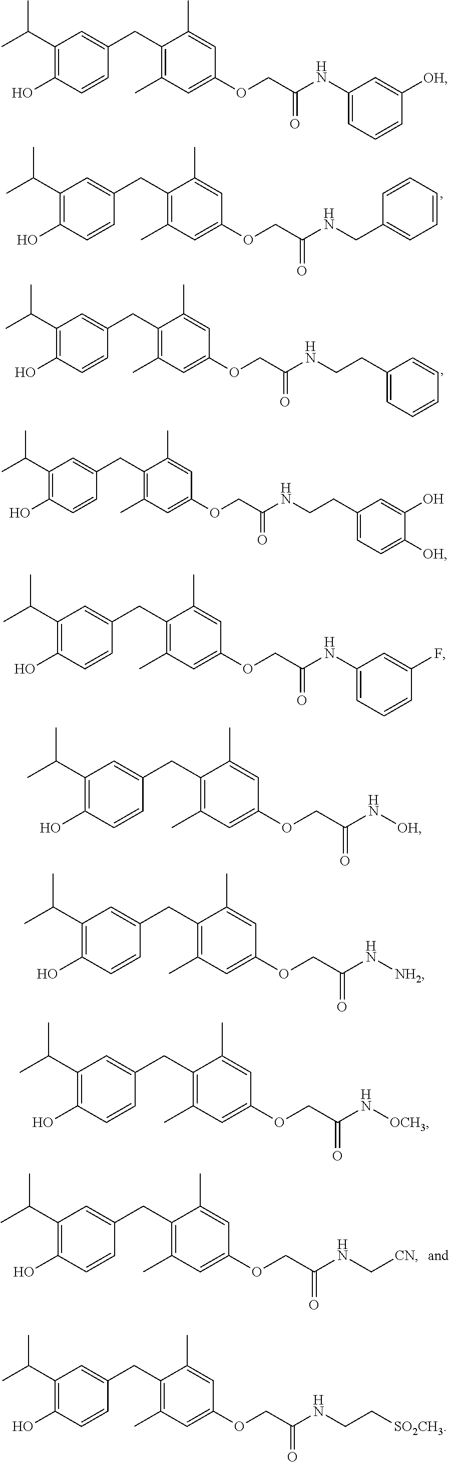

### Pharmaceutical Compositions

In some embodiments described herein is a pharmaceutical composition comprising a fatty acid amide hydrolase (FAAH) cleavable prodrug of Formula (I), (II), (III), or (IV) described herein. In some embodiments described herein is a pharmaceutical composition comprising a fatty acid amide hydrolase (FAAH) cleavable prodrug of Formula (I), (II), (III), or (IV) described herein and a pharmaceutically acceptable excipient. In some embodiments described herein is a pharmaceutical composition comprising a fatty acid amide hydrolase (FAAH) cleavable prodrug of Formula (I) described herein and a pharmaceutically acceptable excipient. In some embodiments described herein is a pharmaceutical composition comprising a fatty acid amide hydrolase (FAAH) cleavable prodrug of Formula (II) described herein and a pharmaceutically acceptable excipient. In some embodiments described herein is a pharmaceutical composition comprising a fatty acid amide hydrolase (FAAH) cleavable prodrug of Formula (III) described herein and a pharmaceutically acceptable excipient. In some embodiments described herein is a pharmaceutical composition comprising a fatty acid amide hydrolase (FAAH) cleavable prodrug of Formula (IV) described herein and a pharmaceutically acceptable excipient.

### Peripherally Restricted FAAH Inhibitors

In some embodiments described herein is a pharmaceutical composition comprising a fatty acid amide hydrolase (FAAH) cleavable prodrug of Formula (I), (II), (III), or (IV) described herein and further comprising a peripherally restricted FAAH inhibitor. In some embodiments, the peripherally restricted FAAH inhibitor is disclosed in US 2008/0306046, which is herein incorporated by reference in its entirety.

In some embodiments, the peripherally restricted FAAH inhibitor is a compound of Formula (X), or a pharmaceutically acceptable salt thereof:

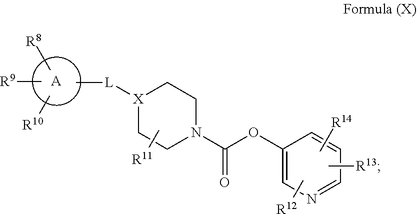

wherein:
ring A is a benzene ring, cyclopentane ring, cyclohexane ring, cycloheptane ring, or a 5- to 7 membered nitrogen-containing hetero ring;
L is a single bond, lower alkylene, lower alkenylene, —N(R¹⁵)—C(═O)—, —C(═O)—N(R¹⁵)—, -(lower alkenylene)-C(═O), —O—, or C(═O);
R¹⁵ is H or lower alkyl;
X is CH or N;
R⁸, R⁹, and R¹⁰ are each independently selected from:
(i) a group selected from the group consisting of H, halo, —CN, CF₃, lower alkyl, and —O-lower alkyl;
(ii) aryl optionally substituted with 1 to 5 groups independently selected from the group consisting of H, halo, —CN, CF₃, lower alkyl, and —O-lower alkyl;
(iii) nitrogen-containing heteroaryl optionally substituted with 1 to 5 groups independently selected from the group consisting of H, halo, —CN, —CF₃, lower alkyl, and —O-lower alkyl;
(iv) R¹⁶-(lower alkenylene)-O—;
(v) R¹⁶-(lower alkenylene)-N(R¹⁵)—; or
(vi) R¹⁷R¹⁸N—C(═O)—;
R¹⁶ is
(i) aryl optionally substituted with 1 to 5 groups independently selected from the group consisting of H, halo, —CN, —CF₃, lower alkyl, and —O-lower alkyl;
(ii) nitrogen-containing heteroaryl optionally substituted with 1 to 5 groups independently selected from the group consisting of H, halo, —CN, —CF₃, lower alkyl, and —O-lower alkyl; or
(iii) 3- to 8-membered cycloalkyl;
R¹⁷ and R¹⁸ are each independently selected from H, lower alkyl, and 3- to 8-membered cycloalkyl; or R¹⁷ and R¹⁸ may form, together with the nitrogen atom bonded thereto, a 3- to 8-membered nitrogen-containing hetero ring;
R¹¹ is selected from H, lower alkyl, and oxo (═O); and one of R¹², R¹³, and R¹⁴ is —C(═O)—O-(lower alkyl) or —CO₂H, and the others are H.

In some embodiments, the peripherally restricted FAAH inhibitor is 5-(((4-(4-((3-fluorobenzyl)oxy)phenoxy)piperidin-1-yl)carbonyl)oxy)nicotinic acid. In some embodiments, the peripherally restricted FAAH inhibitor is 5-(((4-(2-phenylethyl)piperidin-1-yl)carbonyl)oxy)nicotinic acid. In some embodiments, the peripherally restricted FAAH inhibitor is 5-(((4-(4-(2-cyclohexylethoxy)phenoxy)piperidin-1-yl)carbonyl)oxy)nicotinic acid.

In some embodiments, the peripherally restricted FAAH inhibitor is 5-(((4-((E)-2-phenylvinyl)piperidin-1-yl)carbonyl)oxy)nicotinic acid. In some embodiments, the peripherally restricted FAAH inhibitor is 5-(((4-(3-(1-(6-methylpyridin-2-yl)piperidin-4-yl)propyl)piperidin-1-yl)carbonyl)oxy)nicotinic acid. In some embodiments, the peripherally restricted FAAH inhibitor is 5-(methoxycarbonyl)pyridin-3-yl 4-(2-phenylethyl)piperazine-1-carboxylate. In some embodiments, the peripherally restricted FAAH inhibitor is ASP-3652. In some embodiments, the peripherally restricted FAAH inhibitor is ASP-3652 which is 5-(((4-(2-phenylethyl)piperidin-1-yl)carbonyl)oxy)nicotinic acid.

### Excipients

Suitable optional excipients for use in the pharmaceutical compositions described herein include any commonly used excipients in pharmaceutics and are selected on the basis of compatibility with the active pharmaceutical agent and the release profile properties of the desired dosage form. Excipients include, but are not limited to, binders, fillers, flow aids, disintegrants, lubricants, glidants, polymeric carriers, plasticizers, stabilizers, surfactants, and the like. A summary of excipients described herein, may be found, for example in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference in their entirety.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g.

Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinyl pyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinyl pyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

Fillers or diluents increase bulk in the pharmaceutical formulation. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); hydroxypropylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; sodium chloride; inositol; bentonite; and the like.

Glidants improve the flow characteristics of a powder mixtures. Such compounds include, e.g., colloidal silicon dioxide such as Cab-o-sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®) and the like.

Lubricants are compounds which prevent, reduce, or inhibit adhesion or friction of materials. Exemplary lubricants include, e.g., stearic acid; calcium hydroxide, talc; a hydrocarbon such as mineral oil, or hydrogenated vegetable oil such as hydrogenated soybean oil (Sterotex®), Lubritab®, Cutina®; higher fatty acids and their alkali-metal and alkaline earth metal salts, such as aluminum, calcium, magnesium, zinc, stearic acid, sodium stearates, magnesium stearate, glycerol, talc, waxes, Stearowet®, boric acid, sodium acetate, leucine, a polyethylene glycol or a methoxypolyethylene glycol such as Carbowax™, sodium oleate, glyceryl behenate (Compitrol 888®), glyceryl palmitostearate (Precirol®), colloidal silica such as Syloid™, Carb-O-Sil®, a starch such as corn starch, silicone oil, a surfactant, and the like. Hydrophilic lubricants include, e.g., sodium stearyl fumarate (currently marketed under the trade name PRUV®), polyethylene glycol (PEG), magnesium lauryl sulfate, sodium lauryl sulfate (SLS), sodium benzoate, sodium chloride, and the like.

Disintegrants facilitate breakup or disintegration of the pharmaceutical formulation after administration. Examples of disintegrants include a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinyl pyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a resin such as a cation-exchange resin; citrus pulp; sodium lauryl sulfate; sodium lauryl sulfate in combination starch; and the like.

Polymeric carriers include compounds such as polyvinyl pyrrolidone, e.g., polyvinylpolyvinyl pyrrolidone K12, polyvinyl pyrrolidone K17, polyvinyl pyrrolidone K25, or polyvinyl pyrrolidone K30, polyvinyl pyrrolidone vinyl acetate (PVPVA 64), hydroxypropylmethylcellulose (HPMC), hydroxypropylmethylcellulose acetylsuccinate (HPMC AS), and methylmethacrylate polymers (Eudragit polymers) and the like.

Stabilizers include compounds such as any anti-oxidation agents, e.g., butylated hydroxytoluene (BHT), sodium ascorbate, and tocopherol; buffers, acids, and the like.

Surfactants include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), d-α-tocopheryl polyethylene glycol succinate (Vitamin E TPGS); and the like.

The aforementioned excipients are given as examples only and are not meant to include all possible choices. Other suitable excipient classes include coloring agents, granulating agents, preservatives, anti-foaming agents, plasticizers, and the like. Additionally, many excipients can have more than one role or function, or can be classified in more than one group; the classifications are descriptive only, and are not intended to limit any use of a particular excipient.

Disclosed pharmaceutical formulations are administered to patients (animals and humans) in need of such treatment in dosages that will provide optimal pharmaceutical efficacy. It will be appreciated that the dose required for use in any particular application will vary from patient to patient, not only with the particular pharmaceutical formulation selected, but also with the nature of the condition being treated, the age and condition of the patient, concurrent medication or special diets then being followed by the patient, and other factors, with the appropriate dosage ultimately being at the discretion of the attendant physician.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are offered for purposes of illustration and are not intended to limit the scope of the claims provided herein. All literature citations in these examples and throughout this specification are incorporated herein by references for all legal purposes to be served thereby. The starting materials and reagents used for the synthesis of the compounds described herein may be synthesized or can be obtained from commercial sources, such as, but not limited to, Sigma-Aldrich, Acros Organics, Fluka, and Fischer Scientific. In some embodiments, the compounds provided herein are synthesized as described in US 2019/0210950, which is herein incorporated by reference. In some embodiments, the compounds provided herein are synthesized as described in US 2021/0002208, which is herein incorporated by reference. In some embodiments, the compounds provided herein are synthesized as described in WO 2021/108549, which is herein incorporated by reference. In some embodiments, the compounds provided herein are synthesized as described in PCT/US2022/028187, which is herein incorporated by reference.

### Example 1: FAAH Substrate Evaluation

Purified recombinant human FAAH (rhFAAH) was purchased from Cayman Chemical (Ann Arbor, MI, USA). The total volume for each incubation was 400 μL containing a final 0.5 ng/μL rhFAAH, 1 μM test compound, 1.25% ethanol or 1 μM PF-3845 (FAAH inhibitor), and 0.1% bovine serum albumin in Tris-EDTA buffer at pH 8.0). The positive control was LL-341001. The incubation was conducted at the room temperature. At 0, 5, 15, 30 and 60 minutes, an aliquot of 30 μL reaction mixtures was removed and mixed with 300 μL acetonitrile containing 5 ng/mL terfenadine and 10 ng/mL tolbutamide as internal standards to quench the reaction. The resulting mixture was centrifuged at 4000 rpm, 4° C. for 15 minutes, and 100 μL supernatant was ready for LC-MS/MS analysis to measure the formation of acid metabolite.

### LC-MS/MS Analysis

Acquity Ultra Performance LC system from Waters was used for sample analysis. The chromatography was performed on a reverse phase Kinetex 2.6 μm C18 column, 2.1×30 mm, 100 Å. The mobile phase A comprised of 0.1% formic acid in water and mobile phase B comprised of 0.1% formic acid in acetonitrile with a 2-min run time at the flow rate of 0.8 mL/min for the acid metabolite from positive control or a 1.5 min run time at the flow rate of 0.9 mL/min for the acid metabolite of test compounds. The mass spectrometer (API-5500 and API Q Trap 4000 Applied Biosystems/MDS SCIEX Instruments, Framingham, MA, USA) was operated under ESI positive or negative ion MRM mode.

### Data Analysis

The formation of acid metabolite was monitored and quantified using one calibration point of 1 μM. The observed rate constant (ke) for the acid metabolite formation was calculated by plotting the metabolite concentration versus time of incubation with the slope being ke and is shown in Table 1.

TABLE 1

| Compound | Structure | ke |
|---|---|---|
| 1 | 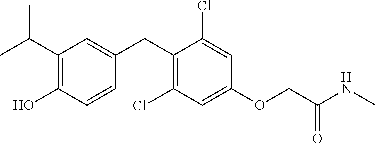 [SMILES: CNC(=O)COc1cc(Cl)c(Cc2ccc(O)c(C(C)C)c2)c(Cl)c1] | A |
| 2 | 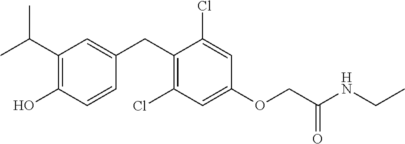 [SMILES: CCNC(=O)COc1cc(Cl)c(Cc2ccc(O)c(C(C)C)c2)c(Cl)c1] | A |
| 3 | 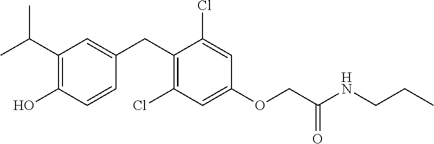 [SMILES: CCCNC(=O)COc1cc(Cl)c(Cc2ccc(O)c(C(C)C)c2)c(Cl)c1] | A |
| 4 | 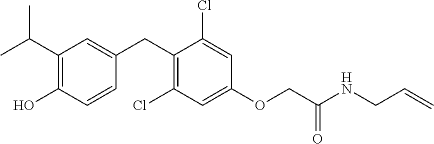 [SMILES: C=CCNC(=O)COc1cc(Cl)c(Cc2ccc(O)c(C(C)C)c2)c(Cl)c1] | A |
| 5 | 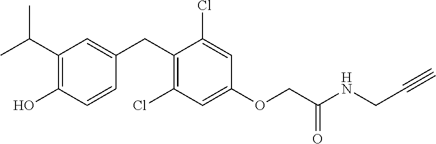 [SMILES: C#CCNC(=O)COc1cc(Cl)c(Cc2ccc(O)c(C(C)C)c2)c(Cl)c1] | A |
| 6 | 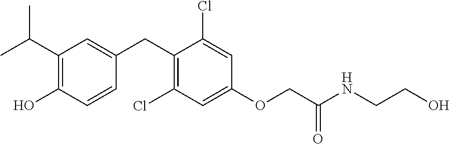 [SMILES: CC(C)c1cc(Cc2c(Cl)cc(OCC(=O)NCCO)cc2Cl)ccc1O] | A |
| 7 | 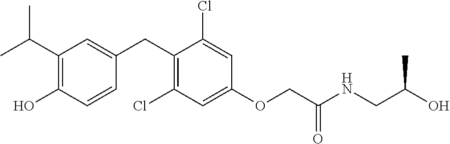 [SMILES: CC(C)c1cc(Cc2c(Cl)cc(OCC(=O)NC[C@@H](C)O)cc2Cl)ccc1O] | B |
| 8 | 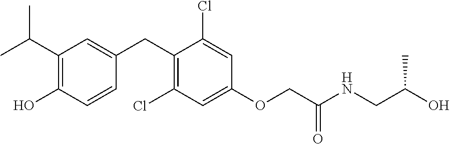 [SMILES: CC(C)c1cc(Cc2c(Cl)cc(OCC(=O)NC[C@H](C)O)cc2Cl)ccc1O] | B |
| 9 | 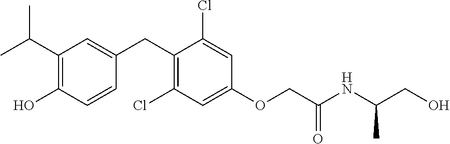 [SMILES: CC(C)c1cc(Cc2c(Cl)cc(OCC(=O)N[C@H](C)CO)cc2Cl)ccc1O] | A |
| 10 | 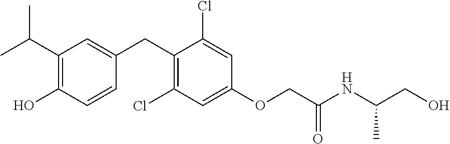 [SMILES: CC(C)c1cc(Cc2c(Cl)cc(OCC(=O)N[C@@H](C)CO)cc2Cl)ccc1O] | A |
| 11 | 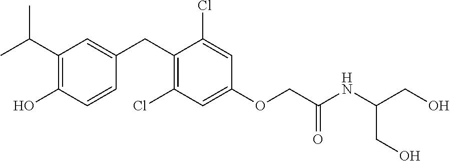 [SMILES: CC(C)c1cc(Cc2c(Cl)cc(OCC(=O)NC(CO)CO)cc2Cl)ccc1O] | B |
| 12 | 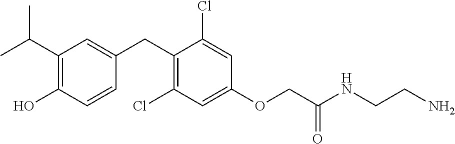 [SMILES: CC(C)c1cc(Cc2c(Cl)cc(OCC(=O)NCCN)cc2Cl)ccc1O] | B |
| 13 | 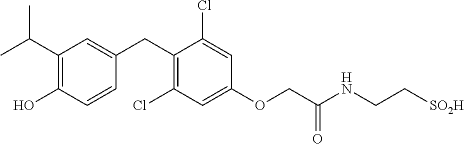 | B |
| 14 | 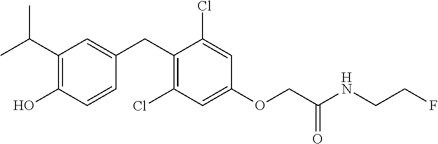 [SMILES: CC(C)c1cc(Cc2c(Cl)cc(OCC(=O)NCCF)cc2Cl)ccc1O] | A |
| 15 | 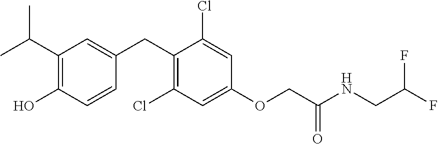 [SMILES: CC(C)c1cc(Cc2c(Cl)cc(OCC(=O)NCC(F)F)cc2Cl)ccc1O] | A |
| 16 | 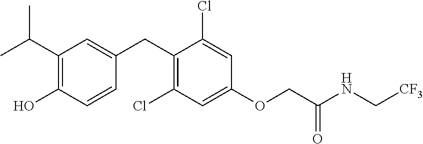 [SMILES: CC(C)c1cc(Cc2c(Cl)cc(OCC(=O)NCC(F)(F)F)cc2Cl)ccc1O] | A |
| 17 | 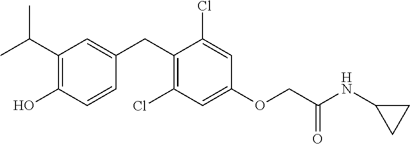 [SMILES: CC(C)c1cc(Cc2c(Cl)cc(OCC(=O)NC3CC3)cc2Cl)ccc1O] | A |
| 18 | 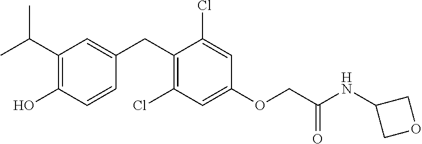 [SMILES: CC(C)c1cc(Cc2c(Cl)cc(OCC(=O)NC3COC3)cc2Cl)ccc1O] | A |
| 19 | 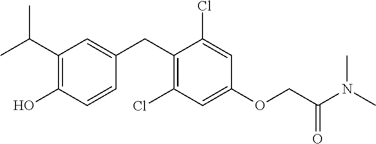 [SMILES: CC(C)c1cc(Cc2c(Cl)cc(OCC(=O)N(C)C)cc2Cl)ccc1O] | B |
| 20 | 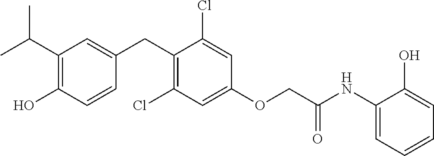 [SMILES: CC(C)c1cc(Cc2c(Cl)cc(OCC(=O)Nc3ccccc3O)cc2Cl)ccc1O] | A |
| 21 | 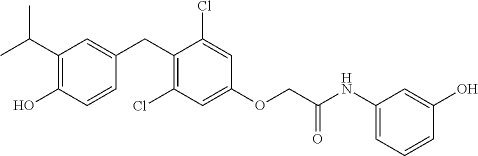 [SMILES: CC(C)c1cc(Cc2c(Cl)cc(OCC(=O)Nc3cccc(O)c3)cc2Cl)ccc1O] | A |
| 22 | 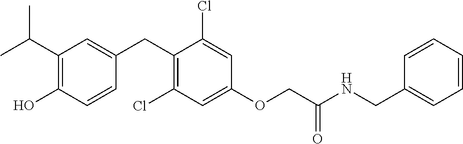 [SMILES: CC(C)c1cc(Cc2c(Cl)cc(OCC(=O)NCc3ccccc3)cc2Cl)ccc1O] | A |
| 23 | 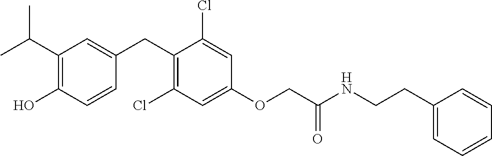 [SMILES: CC(C)c1cc(Cc2c(Cl)cc(OCC(=O)NCCc3ccccc3)cc2Cl)ccc1O] | B |
| 24 | 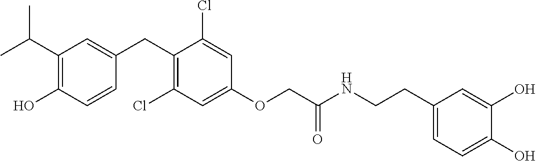 [SMILES: CC(C)c1cc(Cc2c(Cl)cc(OCC(=O)NCCc3ccc(O)c(O)c3)cc2Cl)ccc1O] | B |
| 25 | 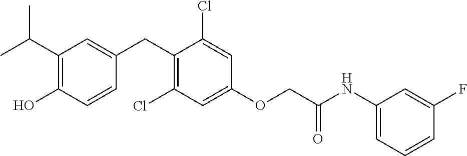 [SMILES: CC(C)c1cc(Cc2c(Cl)cc(OCC(=O)Nc3cccc(F)c3)cc2Cl)ccc1O] | A |
| 26 | 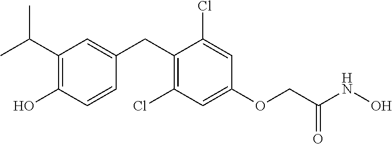 [SMILES: CC(C)c1cc(Cc2c(Cl)cc(OCC(=O)NO)cc2Cl)ccc1O] | A |
| 27 | 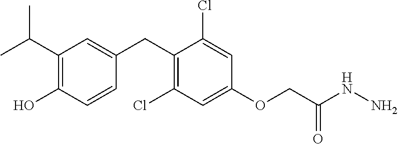 [SMILES: CC(C)c1cc(Cc2c(Cl)cc(OCC(=O)NN)cc2Cl)ccc1O] | A |
| 28 | 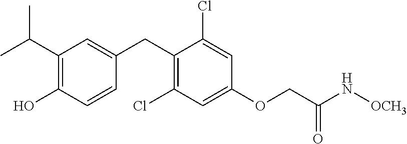 [SMILES: CONC(=O)COc1cc(Cl)c(Cc2ccc(O)c(C(C)C)c2)c(Cl)c1] | A |
| 29 | 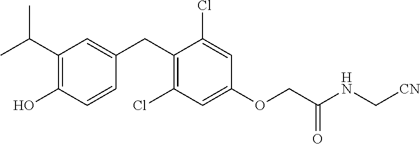 [SMILES: CC(C)c1cc(Cc2c(Cl)cc(OCC(=O)NCC#N)cc2Cl)ccc1O] | A |
| 30 | 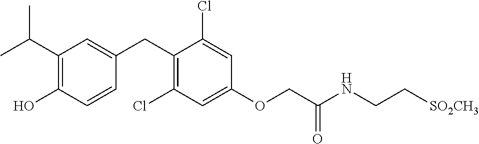 [SMILES: CC(C)c1cc(Cc2c(Cl)cc(OCC(=O)NCCS(C)(=O)=O)cc2Cl)ccc1O] | A |
| 31 | 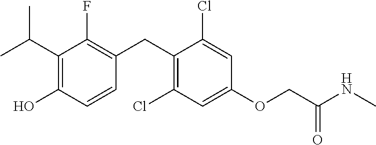 [SMILES: CNC(=O)COc1cc(Cl)c(Cc2ccc(O)c(C(C)C)c2F)c(Cl)c1] | A |
| 32 | 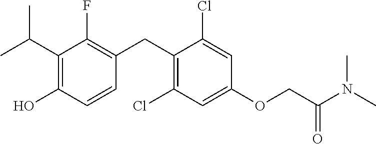 [SMILES: CC(C)c1c(O)ccc(Cc2c(Cl)cc(OCC(=O)N(C)C)cc2Cl)c1F] | A |
| 33 | 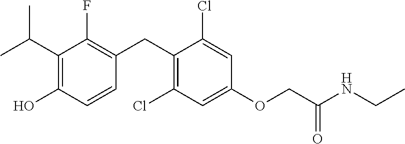 [SMILES: CCNC(=O)COc1cc(Cl)c(Cc2ccc(O)c(C(C)C)c2F)c(Cl)c1] | A |
| 34 | 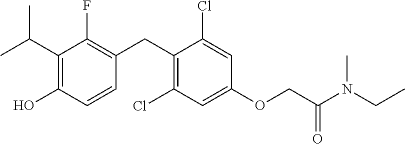 [SMILES: CCN(C)C(=O)COc1cc(Cl)c(Cc2ccc(O)c(C(C)C)c2F)c(Cl)c1] | C |
| 35 | 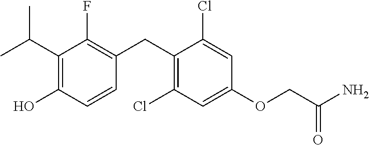 [SMILES: CC(C)c1c(O)ccc(Cc2c(Cl)cc(OCC(N)=O)cc2Cl)c1F] | A |
| 36 | 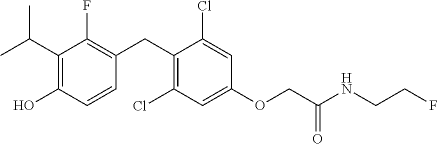 [SMILES: CC(C)c1c(O)ccc(Cc2c(Cl)cc(OCC(=O)NCCF)cc2Cl)c1F] | A |
| 37 | 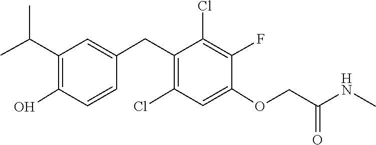 [SMILES: CNC(=O)COc1cc(Cl)c(Cc2ccc(O)c(C(C)C)c2)c(Cl)c1F] | B |
| 38 | 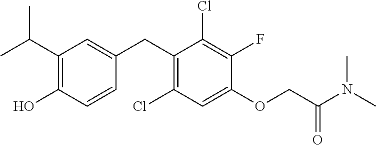 [SMILES: CC(C)c1cc(Cc2c(Cl)cc(OCC(=O)N(C)C)c(F)c2Cl)ccc1O] | B |
| 39 | 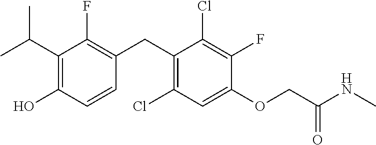 [SMILES: CNC(=O)COc1cc(Cl)c(Cc2ccc(O)c(C(C)C)c2F)c(Cl)c1F] | B |
| 40 | 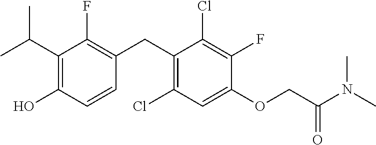 [SMILES: CC(C)c1c(O)ccc(Cc2c(Cl)cc(OCC(=O)N(C)C)c(F)c2Cl)c1F] | B |
| 41 | 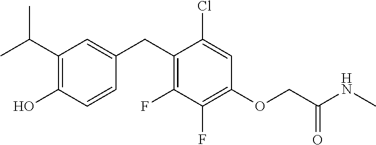 [SMILES: CNC(=O)COc1cc(Cl)c(Cc2ccc(O)c(C(C)C)c2)c(F)c1F] | NT |
| 42 | 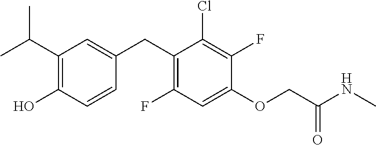 [SMILES: CNC(=O)COc1cc(F)c(Cc2ccc(O)c(C(C)C)c2)c(Cl)c1F] | A |
| 43 | 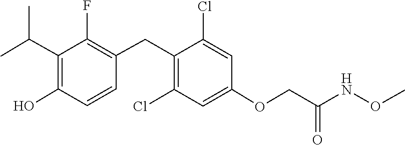 [SMILES: CONC(=O)COc1cc(Cl)c(Cc2ccc(O)c(C(C)C)c2F)c(Cl)c1] | NT |
| 44 | 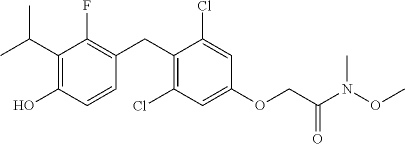 [SMILES: CON(C)C(=O)COc1cc(Cl)c(Cc2ccc(O)c(C(C)C)c2F)c(Cl)c1] | A |
| 45 | 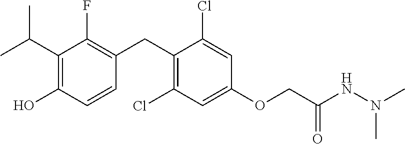 [SMILES: CC(C)c1c(O)ccc(Cc2c(Cl)cc(OCC(=O)NN(C)C)cc2Cl)c1F] | A |
| 101 | 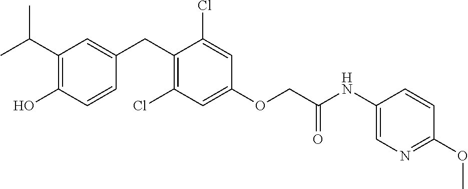 [SMILES: COc1ccc(NC(=O)COc2cc(Cl)c(Cc3ccc(O)c(C(C)C)c3)c(Cl)c2)cn1] | A |
| 102 | 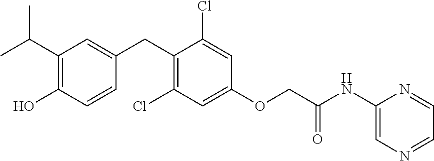 [SMILES: CC(C)c1cc(Cc2c(Cl)cc(OCC(=O)Nc3cnccn3)cc2Cl)ccc1O] | A |
| 103 | 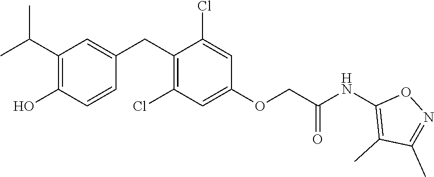 [SMILES: Cc1noc(NC(=O)COc2cc(Cl)c(Cc3ccc(O)c(C(C)C)c3)c(Cl)c2)c1C] | A |
| 104 | 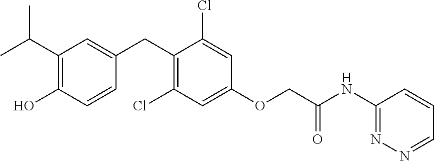 [SMILES: CC(C)c1cc(Cc2c(Cl)cc(OCC(=O)Nc3cccnn3)cc2Cl)ccc1O] | A |
| 105 | 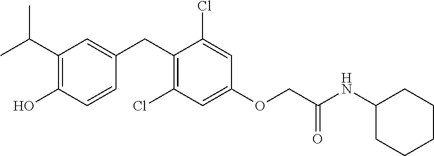 [SMILES: CC(C)c1cc(Cc2c(Cl)cc(OCC(=O)NC3CCCCC3)cc2Cl)ccc1O] | C |
| 106 | 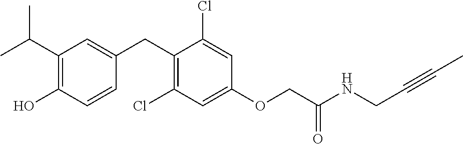 [SMILES: CC#CCNC(=O)COc1cc(Cl)c(Cc2ccc(O)c(C(C)C)c2)c(Cl)c1] | B |
| 107 | 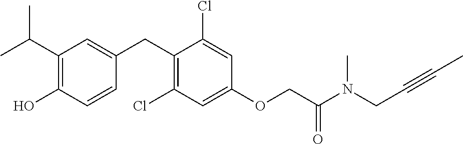 [SMILES: CC#CCN(C)C(=O)COc1cc(Cl)c(Cc2ccc(O)c(C(C)C)c2)c(Cl)c1] | B |
| 108 | 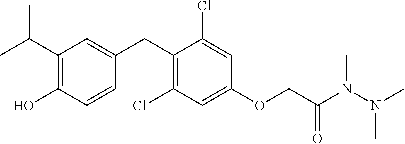 [SMILES: CC(C)c1cc(Cc2c(Cl)cc(OCC(=O)N(C)N(C)C)cc2Cl)ccc1O] | B |
| 109 | 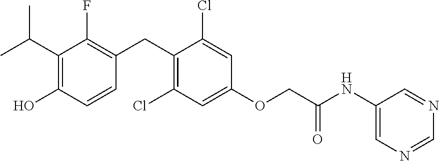 [SMILES: CC(C)c1c(O)ccc(Cc2c(Cl)cc(OCC(=O)Nc3cncnc3)cc2Cl)c1F] | A |
| 110 | 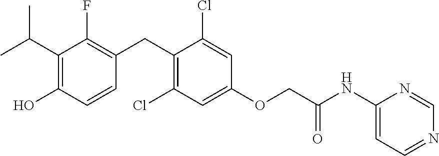 [SMILES: CC(C)c1c(O)ccc(Cc2c(Cl)cc(OCC(=O)Nc3ccncn3)cc2Cl)c1F] | A |
| 111 | 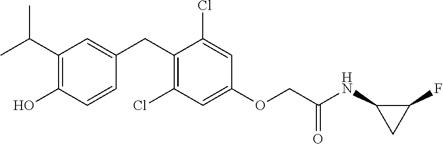 [SMILES: CC(C)c1cc(Cc2c(Cl)cc(OCC(=O)N[C@@H]3C[C@@H]3F)cc2Cl)ccc1O] | A |
| 112 | 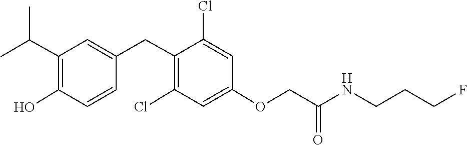 [SMILES: CC(C)c1cc(Cc2c(Cl)cc(OCC(=O)NCCCF)cc2Cl)ccc1O] | A |
| 113 | 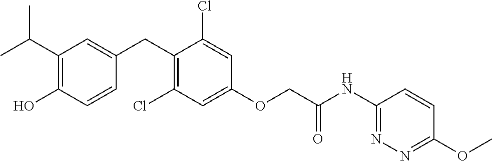 [SMILES: COc1ccc(NC(=O)COc2cc(Cl)c(Cc3ccc(O)c(C(C)C)c3)c(Cl)c2)nn1] | A |
| 114 | 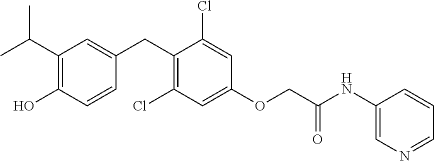 [SMILES: CC(C)c1cc(Cc2c(Cl)cc(OCC(=O)Nc3cccnc3)cc2Cl)ccc1O] | A |
| 115 | 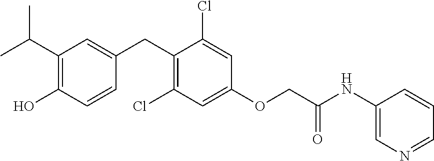 [SMILES: CC(C)c1cc(Cc2c(Cl)cc(OCC(=O)Nc3cccnc3)cc2Cl)ccc1O] | A |
| 116 | 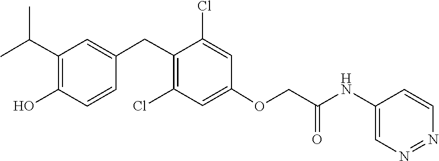 [SMILES: CC(C)c1cc(Cc2c(Cl)cc(OCC(=O)Nc3ccnnc3)cc2Cl)ccc1O] | A |
| 117 | 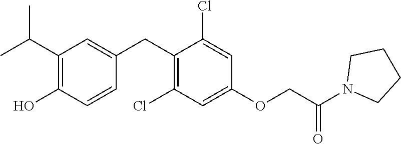 [SMILES: CC(C)c1cc(Cc2c(Cl)cc(OCC(=O)N3CCCC3)cc2Cl)ccc1O] | B |
| 118 | 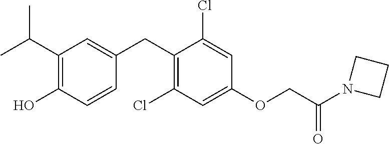 [SMILES: CC(C)c1cc(Cc2c(Cl)cc(OCC(=O)N3CCC3)cc2Cl)ccc1O] | B |
| 119 | 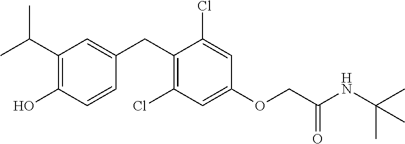 [SMILES: CC(C)c1cc(Cc2c(Cl)cc(OCC(=O)NC(C)(C)C)cc2Cl)ccc1O] | C |
| 120 | 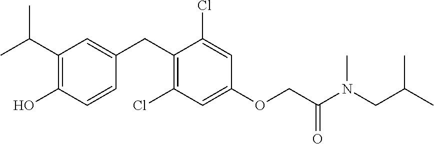 [SMILES: CC(C)CN(C)C(=O)COc1cc(Cl)c(Cc2ccc(O)c(C(C)C)c2)c(Cl)c1] | C |
| 121 | 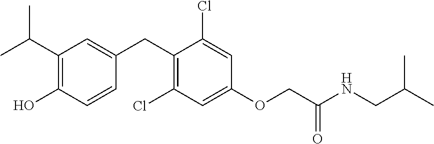 [SMILES: CC(C)CNC(=O)COc1cc(Cl)c(Cc2ccc(O)c(C(C)C)c2)c(Cl)c1] | C |
| 122 | 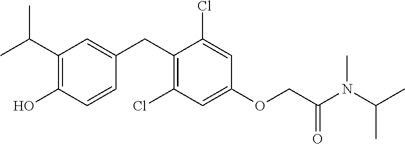 [SMILES: CC(C)c1cc(Cc2c(Cl)cc(OCC(=O)N(C)C(C)C)cc2Cl)ccc1O] | B |
| 123 | 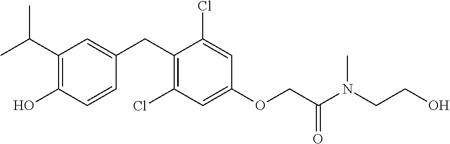 [SMILES: CC(C)c1cc(Cc2c(Cl)cc(OCC(=O)N(C)CCO)cc2Cl)ccc1O] | B |
| 124 | 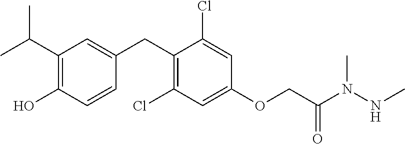 [SMILES: CNN(C)C(=O)COc1cc(Cl)c(Cc2ccc(O)c(C(C)C)c2)c(Cl)c1] | B |
| 125 | 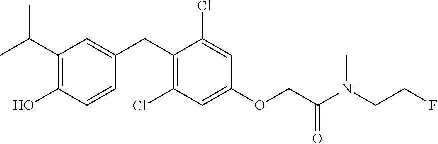 [SMILES: CC(C)c1cc(Cc2c(Cl)cc(OCC(=O)N(C)CCF)cc2Cl)ccc1O] | B |
| 126 | 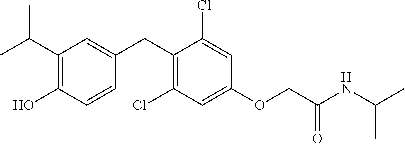 [SMILES: CC(C)NC(=O)COc1cc(Cl)c(Cc2ccc(O)c(C(C)C)c2)c(Cl)c1] | A |
| 127 | 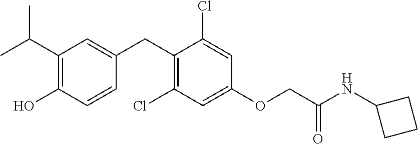 [SMILES: CC(C)c1cc(Cc2c(Cl)cc(OCC(=O)NC3CCC3)cc2Cl)ccc1O] | B |
| 128 | 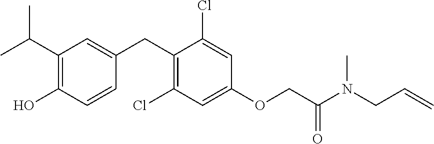 [SMILES: C=CCN(C)C(=O)COc1cc(Cl)c(Cc2ccc(O)c(C(C)C)c2)c(Cl)c1] | B |
| 129 | 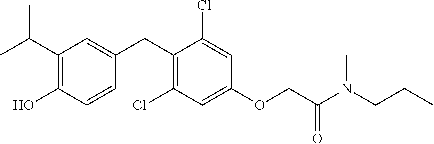 [SMILES: CCCN(C)C(=O)COc1cc(Cl)c(Cc2ccc(O)c(C(C)C)c2)c(Cl)c1] | B |
| 130 | 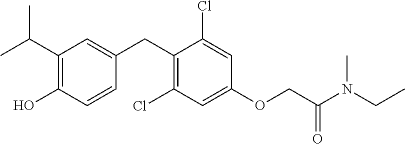 [SMILES: CCN(C)C(=O)COc1cc(Cl)c(Cc2ccc(O)c(C(C)C)c2)c(Cl)c1] | B |
| 131 | 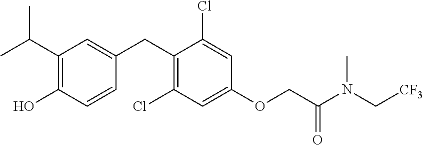 [SMILES: CC(C)c1cc(Cc2c(Cl)cc(OCC(=O)N(C)CC(F)(F)F)cc2Cl)ccc1O] | B |
| 132 | 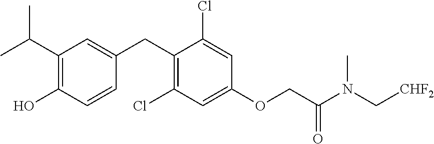 [SMILES: CC(C)c1cc(Cc2c(Cl)cc(OCC(=O)N(C)CC(F)F)cc2Cl)ccc1O] | B |
| 133 | 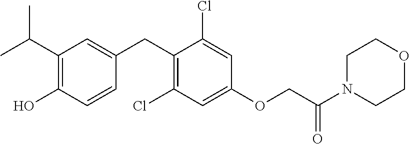 [SMILES: CC(C)c1cc(Cc2c(Cl)cc(OCC(=O)N3CCOCC3)cc2Cl)ccc1O] | A |

A = ke is more than or equal to 0.1;
B = ke is less than 0.1 and more than 0;
C = ke is 0;
NT = not tested.

### Example 2: In Vitro Prodrug and Agonist TRP Receptor Selectivity

LL-341070 and LL-341070A (LL-341070 is Compound 31 described herein, which is activated to LL-341070 Å; LL-341070A is 2-(3,5-dichloro-4-(2-fluoro-4-hydroxy-3-isopropylbenzyl)phenoxy)acetic acid) were evaluated for potency and selectivity for the thyroid hormone beta receptor (TRβ3). LL-341070 is a thyromimetic prodrug of Formula (I) described herein. Following fatty acid amide hydrolase (FAAH)-mediated conversion, LL-341070 delivers LL-341070 Å, a potent and selective small molecule agonist of thyroid hormone receptor (TR) beta. In vitro potency was determined via test compounds administered to luciferase-based TR reporter cell lines, using thyroid hormone (T3) as a positive control. Table 2 depicts the potency profiles of LL-341070 prodrug and LL-341070A active metabolite against TRβ and TRα as measured in half maximal effective concentration (EC50), with selectivity measurement adjusted for the TRα-bias of T3 in the assays. Both LL-341070 and LL-341070A show enhanced selectivity for TRβ, with LL-341070A showing enhanced potency.

TABLE 2

| | Prodrug | Active |
|---|---|---|
| Profile | LL-341070 (nM) | L-341070A (nM) |
| TRβ EC₅₀ | 478 | 7.3 |
| TRα EC₅₀ | >10,000 | 24 |
| Selectivity* | n/a | 9.1 |

### Example 3: Engagement of TRP in Brain Increases Expression of T3-Target Genes In Vivo

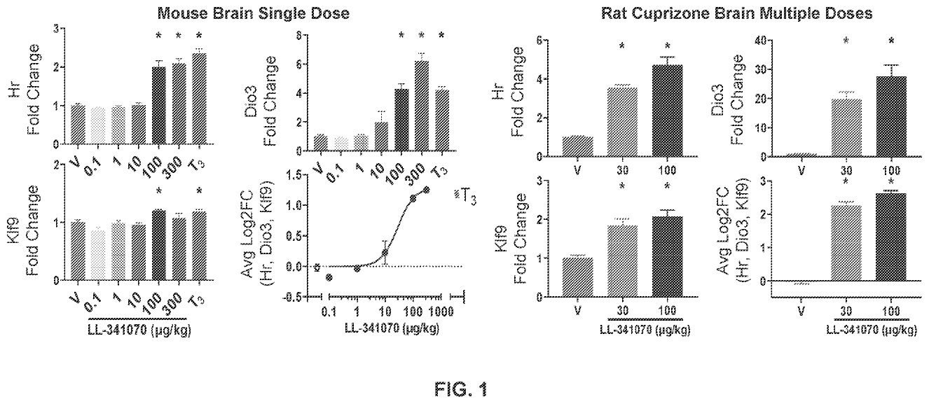

FIG. 1 depicts TRβ target engagement in brain is demonstrated by increased expression of T3-responsive target genes in vivo. Single PO administration of LL-341070 (ranging from about 0.1 μg/kg to about 300 μg/kg) or T3 (about 300 μg/kg) in male C₅₇BL/6 mouse increases expression of Hr, Dio3, Klf9 (quantified by QuaniPlex) and composite average log 2 fold change in brain. Klf9, a T3-responsive gene linked to myelin regeneration in vitro, is upregulated at various treatment concentrations. This expression increase was confirmed in the brain of a rat cuprizone model (as previously discussed) with 21 day repeat administration of LL-341070 at 30 μg/kg or 100 μg/kg, or T3 dosed at 300 μg/kg (quantified by Nanostring). Interestingly, Dio3 has an enhanced expression increase with repeat dosing.

### Example 4: In Vivo Tissue Distribution Demonstrates Enhanced Brain Exposure of Active Compound Compared to Prodrug

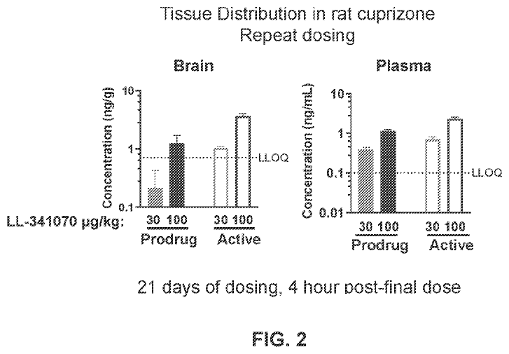

In vivo brain exposure of active compound compared to prodrug was assessed via tissue distribution (TD) assay in mouse and rat cuprizone model, measured as brain exposure ratio of brain to plasma following thyromimetic treatment. As shown in Table 3, single PO administration of LL-341070 (100 μg/kg) or LL-341070A (100 μg/kg), in male C57BL/6 mouse measured in brain and plasma, demonstrates enhanced brain exposure of active compound LL-341070A compared to prodrug LL-341070, leading to a brain-to-plasma AUC ratio >1 for LL-341070 Å, wherein AUC is 0-24 hr. Data shows AUC of LL-341070A in brain is ˜7-fold higher than prodrug LL-341070. Table 3 also depicts brain-to-plasma AUC ratio. As shown in FIG. 2, 21 days of repeat administration of LL-341070 (30 μg/kg or 100 μg/kg) or LL-341070 Å(30 μg/kg or 100 μg/kg), in rat cuprizone model measured in brain and plasma 4 hours post-final dose, demonstrates enhanced brain exposure of active compound LL-341070A compared to prodrug LL-341070.

TABLE 3

| Prodrug | | | |
|---|---|---|---|
| @ 100 μg/kg | Brain AUC | Plasma AUC | Brain/Plasma |
| PO | (ng/mL*h) | (ng/mL*h) | AUC Ratio |
| Prodrug | 10.9 | 18.8 | 0.58 |
| LL-341070 | | | |
| Active | 73.9 | 35.8 | 2.06 |
| LL-341070A | | | |

### Example 5: FAAH Expression is Enriched in Brain

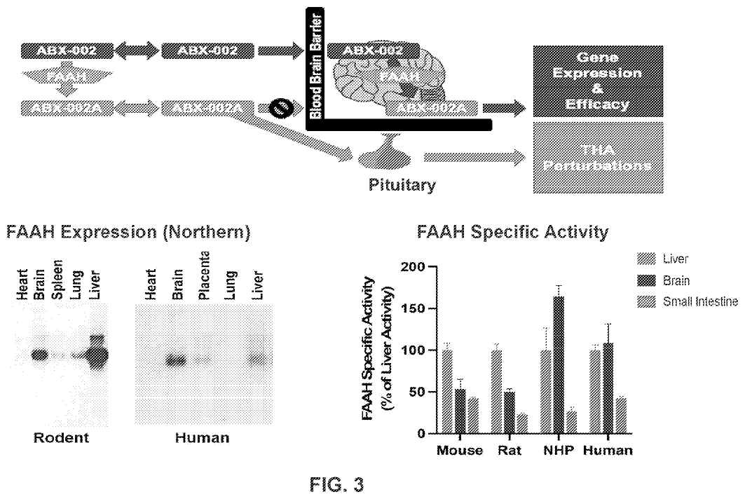

As shown in FIG. 3, brain-directed thyromimetic prodrugs (such as ABX-002 which is Compound 1 described herein, activated to ABX-002 Å) that are activated by fatty acid amide hydrolase (FAAH) were utilized to elucidate mechanisms by which thyromimetics disrupt the thyroid hormone axis (THA). The delivery of potent thyromimetics was altered to help identify whether feedback control on THA derives from central (hypothalamic) or peripheral (pituitary) mechanisms and potentially enhance therapeutic index of thyromimetics. These studies were performed using recombinant FAAH, tissue-derived S9 fractions, in vivo tissue distribution (TD), gene expression in brain and liver, and effects on T₄ in mice as a marker of THA disruption. Northern blot assay confirmed FAAH expressed across species (rodent and human), with relative mRNA FAAH expression enhanced in the brain. FAAH specific activity (cleavage of AMC assay) from tissue-derived S9 fractions of different organs (liver, brain, small intestine) across species (mouse, rat, non-human primate, human), calculated as a percentage of liver activity, was shown to be increased in brain of human and non-human primate.

### Example 6: FAAH Expression Enhances Delivery of ABX-002A to Brain

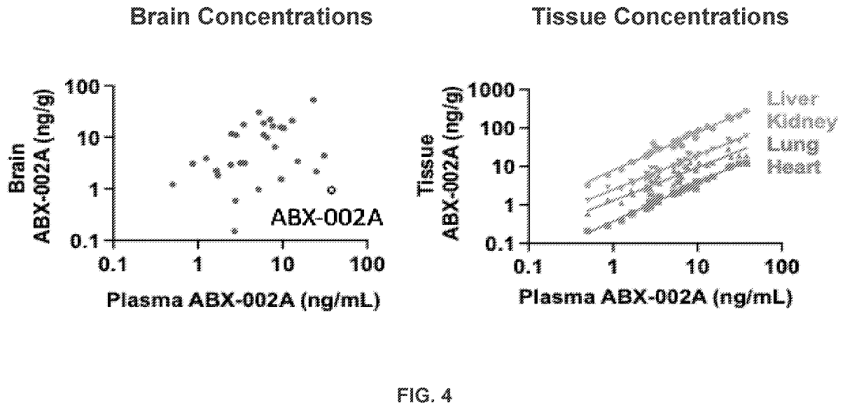

To assess delivery, concentrations of ABX-002A in brain, liver, kidney, lung, and heart were measured 1 hour after SC administration of 30 different prodrugs of ABX-002 Å. As shown in FIG. 4, brain-to-plasma ratios were increased relative to ABX-002A for the prodrugs, while tissue-to-plasma ratios for peripheral organs (liver, kidney, lung, and heart) showed a linear (constant) tissue-to-plasma relationship. Data shows FAAH is highly expressed in the CNS and ABX prodrugs enhance delivery of active metabolite to the brain by >30× with brain-to-plasma ratios >1. In organs other than the brain, data shows tissue concentrations are driven by plasma concentrations of the active metabolite ABX-002A.

### Example 7: Global and Peripheral FAAH Inhibitors Alter Metabolite Distribution in Mice

The ability of globally-penetrant and peripherally-restricted FAAH inhibitors (GFI & PFI, respectively) to alter distribution of ABX-002 and ABX-002A was assessed. Table 4 depicts the potency profiles (measured in apparent IC₅₀s (nM)) of peripheral and global FAAH inhibitors: LL-650177 (PFI), URB9373 (PFI), and PF-044578454 (GFI) obtained after 30 min preincubation with recombinant human FAAH and 7-amino-4-methylcourmarin (AMC).

TABLE 4

| FAAH inhibitor | Apparent IC₅₀s (nM) | Distribution |
|---|---|---|
| LL-650177 | 9.1 | Peripheral |
| URB937 | 69 | Peripheral |
| PF-04457845 | 3.0 | Global |

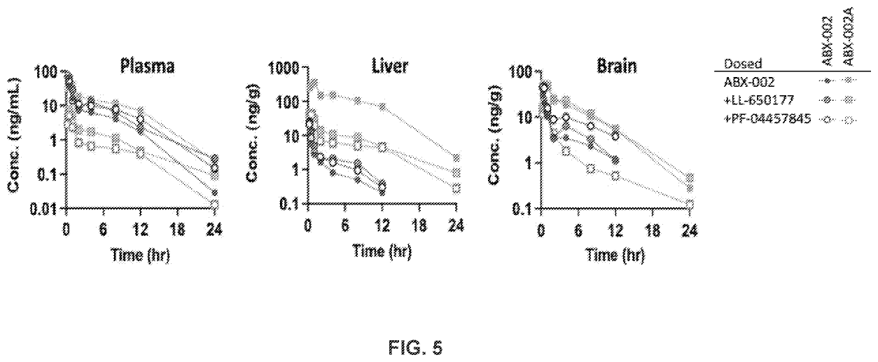

FIG. 5 shows plasma, liver, and brain concentrations after co-dosing prodrug (ABX-002) with or without PFI or GFI. Prodrug levels did not change or slightly increased with FAAH inhibition. Active metabolite (ABX-002A) levels decreased in plasma & liver with PFI & in all organs with GFI. Table 5 depicts the inhibition of active metabolite (LL-650177 or PF-044578454) in AUC in the plasma, liver, and brain after prodrug (ABX-002) co-dosing. Tissue distribution studies in mice confirm global & peripheral inhibition of FAAH.

TABLE 5

| FAAH inhibitor | Plasma | Liver | Brain |
|---|---|---|---|
| LL-650177 | 89% | 91% | −12% |
| PF-04457845 | 94% | 94% | 83% |

### Example 8: Induction of T3-Regulated Genes in View of Prodrug and FAAH Inhibitors

Female C57BL/6 mice (n=5/group), 6-8 weeks old, were acclimated to the study room for at least 3 days before dose administration in the studies. Non-fasted mice were given a single dose of PFI or vehicle orally (PO) on Day 0 at time=−1 hour. Single dose administered at 5 mL/kg based on most recent body weight, collected once for the duration of the study. Following the PFI or vehicle dose, animals were given a single dose of test article at time=0 hour. One group (n=5) was PO administered 300 μg/kg of T3 only at time=0 hours. Approximately 4 hours post test article dose (t=4 hours), animals were humanely euthanized and brain, liver, heart, pituitary, spinal cord, and plasma samples were harvested.

### Sample Processing

a. Expression Analysis Samples—At endpoint, multiple organs were harvested and tissues processed immediately as described below.
i. Brain: For each mouse, the cranium was opened and the brain removed. The cerebellum was sectioned away and the cerebral cortex was hemisected sagittally and the left half collected. After rinsing extraneous blood from the tissue with ice cold 0.9% NaCl, the cerebral cortical specimen was placed into a tube containing 1.2 mL pre-chilled RNALater and stored at 4° C.
ii. Liver: For each mouse, one liver biopsy (100-150 mg) was collected from the left lateral liver lobe. After rinsing extraneous blood from the biopsy with ice cold 0.9% NaCl, the sample was placed into 1.2 mL pre-chilled RNALater and stored at 4° C.
iii. Left Ventricle: For each mouse, the left ventricle (LV) blood cleared using PBIs standard methods, and half of LV free wall was collected. After rinsing extraneous blood from the tissue with ice cold 0.9% NaCl, the LV free wall was placed into 1.2 mL pre-chilled RNALater and stored at 4° C. LV tissue was retained at PBI for potential future analyses or until appropriate genes can be identified for up to 6 months following conclusion of the in-life phase of study. Sample disposition was confirmed prior to disposal.
iv. Pituitary Gland: For each mouse, after removing the brain the pituitary gland was harvested. After rinsing extraneous blood from the pituitary with ice cold 0.9% NaCl, the specimen was placed into 0.15 mL pre-chilled RNALater and stored at 4° C. Pituitary tissue was retained at PBI for potential future analyses or until appropriate genes can be identified for up to 6 months following conclusion of the in-life phase of study. Sample disposition was confirmed prior to disposal.
b. Pharmacokinetic Samples—At endpoint, blood and tissue specimens were processed immediately as described below. Samples for PK analysis were retained at PBI at −80° C. for up to 90 days following conclusion of the in-life phase of study.
i. Plasma: whole blood (˜300 μL) was collected on K3EDTA via cardiac puncture under isoflurane anesthesia. Blood was immediately placed on wet ice. Following the conclusion of takedown procedures, blood was centrifuged at 4° C. for 10 minutes at 10,000×g. Plasma (˜125 μL) was aliquoted to appropriately labelled tubes and flash frozen.
ii. Liver: For each mouse, one liver biopsy (30-50 mg) was collected from the left lateral liver lobe. After rinsing extraneous blood from the biopsy with ice cold 0.9% NaCl, the sample was placed into an appropriately labelled tube and flash-frozen in liquid nitrogen.
iii. Brain: For each mouse, a mid-brain biopsy (30-50 mg) was collected from the right cerebral cortex. After rinsing extraneous blood from the tissue with ice cold 0.9% NaCl, the biopsy was placed into an appropriately labelled tube and flash frozen.
iv. Left Ventricle: For each mouse, the left ventricle (LV) will have blood cleared using PBIs standard methods, and half LV free wall was collected. After rinsing extraneous blood from the tissue with ice cold 0.9% NaCl, the LV free wall was placed into an appropriately labelled tube and flash frozen.

### Target Engagement

Changes in the expression of select genes identified through transcriptomic analysis were measured from purified RNA using a hybridization-based in situ RNA quantification method (NanoString, Seattle, WA). Briefly, fresh tissues were collected in RNALater™ Stabilization Solution, catalog #AM7021 (ThermoFisher Scientific; Carlsbad, CA) and frozen at −20° C. until ready for RNA extraction. Whole blood was collected in MiniCollect K2EDTA tubes, catalog #450480 Greinder Bio-one GmbH (Kremsmunster, Austria), via terminal cardiac puncture and processed to plasma by centrifuging at 2000×g for 10 minutes at 4° C. For RNA extraction, tissues were homogenized using a bead homogenizer in TRIzol Reagent, catalog #15596026 (ThermoFisher Scientific), and RNA was extracted according to manufacturer's protocols and purified using Econospin RNA Mini Spin Columns for RNA (Ephoch Life Sciences, Missouri City, TX, catalog #1940-250) following manufacturer's protocols. Specific gene probes were designed by NanoString Bioinformatics Team using an identified target sequence based on the NCBI Reference Sequence (RefSeq) database. Custom probes were synthesized by Integrated DNA Technologies (IDT; Coralville, IA). mRNA expression was analyzed on an nCounter® SPRINT Profiler NanoString system using a multiplexing approach with nCounter PlexSet-12 Reagent Pack, catalog #PS-GX-PTK-12 (CSO) according to manufacturer's protocols (NanoString, Inc, Seattle, WA).

### Data Analysis

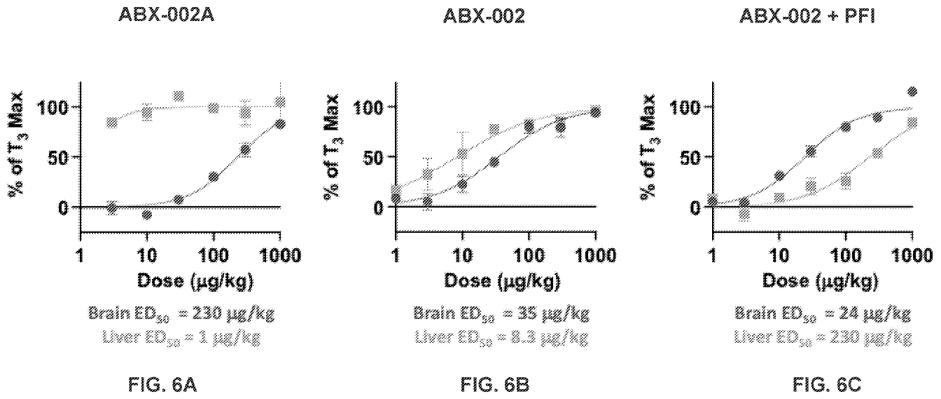

T3-target genes are increased after a single administration of drug with the relative activity in brain vs. liver determined by prodrug and/or FAAH inhibition. Relative activity in brain vs. liver (as a marker of peripheral activity) shifts >1500-fold across the different dosing paradigms. FIGS. 6A, 6B, and 6C show induction of T3-regulated genes in brain (blue) & liver (orange) 4 h after single administration of (A) active metabolite or (B) prodrug alone or (C) prodrug+PFI (URB937). RNA analyzed by Nanostring; Mean fold change of multiple genes was calculated on a log 2 scale and normalized to data obtained for 300 mg/kg of T3. PFI administration reduced potency of prodrugs on activation of T₃-regulated genes in the liver by >10×, without affecting activity or exposure in the brain. PFIs also decreased potency on the THA, consistent with negative feedback based on circulating peripheral metabolite rather than brain exposure. Thus, use of a PFI allowed separation of on-target brain effects from those on THA.

### Example 9: T4 Parallels Peripheral Activity

Female C57BL/6 mice (n=5/group), 6-8 weeks old, were acclimated to the study room for at least 3 days before dose administration in the studies. Mice were dosed at 5 mL/kg based on most recent body weight, collected once for the duration of the study. Based on most recent body weight, collected once for the duration of the study, mice were placed into weight-matched treatment dosing cohorts. Mice were given a single administration of PFI or vehicle orally (PO) daily (n=5/group) for 7 days at time=−1 hour. Following the PFI (100 μg/kg) or vehicle dose (10 mL/kg, p.o.), animals were given test article daily at time=0 hour. Test article administration one of eight dose levels (0.1, 0.3, 1, 3, 10, 30, 100 or 300 μg/kg) on Days 1-7 for a total of seven doses. Mice were dosed PO, QD for 7 days with (A) active metabolite or (B) prodrug alone; (C) prodrug+PFI (LL-650177) or (D) prodrug+GFI. Approximately 4 or 8 hours post test article dose (t=4 hours or t=8 hours), animals were humanely euthanized using standard procedures, and brain, liver, and plasma samples were harvested. RNA from samples harvested 4 hours after final dose was quantified using a hybridization-based in situ RNA quantification method (NanoString, Seattle, WA), as described below. RNA from samples harvested 8 hours after final dose was quantified using a hybridization-based in situ RNA quantification method (QuantiGene Plex), as described below. On the final day of dosing, mice were dosed on a timetable to mitigate the influence of diurnal effects on thyroid hormone sensitive gene expression. Thus, treatment groups were balanced for “time of day” at endpoint sacrifice. Mice were anesthetized 4 or 8 hours after final dosing, have blood collected via retro-orbital puncture, and euthanized using standard procedures. Immediately following euthanasia, tissues were harvested and processed per the following procedures.

### Sample Processing

a. Expression Analysis Samples—At endpoint, multiple organs were harvested and tissues processed immediately as described below.
i. Brain: For each mouse, the cranium was opened and the brain removed. The cerebellum was sectioned away and the cerebral cortex was hemisected sagittally and the left half collected. After rinsing extraneous blood from the tissue with ice cold 0.9% NaCl, the cerebral cortical specimen was placed into a tube containing 1.2 mL pre-chilled RNALater and stored at 4° C.
ii. Liver: For each mouse, one liver biopsy (100-150 mg) was collected from the left lateral liver lobe. After rinsing extraneous blood from the biopsy with ice cold 0.9% NaCl, the sample was placed into 1.2 mL pre-chilled RNALater and stored at 4° C.
iii. Left Ventricle: For each mouse, the left ventricle (LV) blood cleared using PBIs standard methods, and half of LV free wall was collected. After rinsing extraneous blood from the tissue with ice cold 0.9% NaCl, the LV free wall was placed into 1.2 mL pre-chilled RNALater and stored at 4° C. LV tissue was retained at PBI for potential future analyses or until appropriate genes can be identified for up to 6 months following conclusion of the in-life phase of study. Sample disposition was confirmed prior to disposal.
iv. Pituitary Gland: For each mouse, after removing the brain the pituitary gland was harvested. After rinsing extraneous blood from the pituitary with ice cold 0.9% NaCl, the specimen was placed into 0.15 mL pre-chilled RNALater and stored at 4° C. Pituitary tissue was retained at PBI for potential future analyses or until appropriate genes can be identified for up to 6 months following conclusion of the in-life phase of study. Sample disposition was confirmed prior to disposal.
b. Pharmacokinetic Samples—At endpoint, blood and tissue specimens were processed immediately as described below. Samples for PK analysis were retained at PBI at −80° C. for up to 90 days following conclusion of the in-life phase of study.
i. Plasma: whole blood (˜300 μL) was collected on K3EDTA via cardiac puncture under isoflurane anesthesia. Blood was immediately placed on wet ice. Following the conclusion of takedown procedures, blood was centrifuged at 4° C. for 10 minutes at 10,000×g. Plasma (˜125 μL) was aliquoted to appropriately labelled tubes and flash frozen.
ii. Liver: For each mouse, one liver biopsy (30-50 mg) was collected from the left lateral liver lobe. After rinsing extraneous blood from the biopsy with ice cold 0.9% NaCl, the sample was placed into an appropriately labelled tube and flash-frozen in liquid nitrogen.
iii. Brain: For each mouse, a mid-brain biopsy (30-50 mg) was collected from the right cerebral cortex. After rinsing extraneous blood from the tissue with ice cold 0.9% NaCl, the biopsy was placed into an appropriately labelled tube and flash frozen.
iv. Left Ventricle: For each mouse, the left ventricle (LV) will have blood cleared using PBIs standard methods, and half LV free wall was collected. After rinsing extraneous blood from the tissue with ice cold 0.9% NaCl, the LV free wall was placed into an appropriately labelled tube and flash frozen.

### Target Engagement

Tissue samples were prepared for biochemical analysis by cryopowdering on liquid nitrogen, and lysed using PBI's standard methods. Changes in the expression of select genes identified through transcriptomic analysis (mRNA expression) were measured from purified RNA using a hybridization-based in situ RNA quantification method (NanoString or QuantiGene Plex). Target gene expression data was presented as a ratio to the geometric mean of appropriately expressed normalization genes. Briefly, fresh tissues were collected in RNALater™ Stabilization Solution, catalog #AM7021 (ThermoFisher Scientific; Carlsbad, CA) and frozen at −20° C. until ready for RNA extraction. Whole blood was collected in MiniCollect K2EDTA tubes, catalog #450480 Greinder Bio-one GmbH (Kremsmunster, Austria), via terminal cardiac puncture and processed to plasma by centrifuging at 2000×g for 10 minutes at 4° C. For RNA extraction, tissues were homogenized using a bead homogenizer in TRIzol Reagent, catalog #15596026 (ThermoFisher Scientific), and RNA was extracted according to manufacturer's protocols and purified using Econospin RNA Mini Spin/Columns for RNA (Ephoch Life Sciences, Missouri City, TX, catalog #1940-250) following manufacturer's protocols. Specific gene probes were designed by NanoString Bioinformatics Team using an identified target sequence based on the NCBI Reference Sequence (RefSeq) database. contains. Custom probes were synthesized by Integrated DNA Technologies (IDT; Coralville, IA). mRNA expression was analyzed on an nCounter® SPRINT Profiler NanoString system using a multiplexing approach with nCounter PlexSet-12 Reagent Pack, catalog #PS-GX-PTK-12 (CSO) according to manufacturer's protocols (NanoString, Inc, Seattle, WA).

### T4 Analysis

T4 was measured in terminal plasma samples using an ELISA kit (Biovision, Inc., Thyroxine [T4][Mouse/Rat] ELISA Kit, Cat #: K7421-100). Assays were performed according to manufacturer's instructions with minor modifications based on previous assay validation efforts. Briefly, a seven-point standard curve of provided T4 diluted in Assay Buffer (25, 15, 10, 5, 2, 1 μg/dL) was prepared in duplicate for each assay. Plasma samples (undiluted), blanks (Assay Buffer) and standards were added to separate wells of a 96-well plate pre-coated with a T4 capture antibody, followed by addition of T4 Enzyme Conjugate to each well. Plates were then gently shaken (600 rpm) for 20-30 s to mix, and then covered with an acetate plate seal and incubated for 1 h at room temperature (RT) with gentle shaking (600 rpm). Plate contents were aspirated and washed three times with 1× Wash Buffer, then blotted on paper towels to remove excess liquid. TMB Substrate was then added to each well and plates were secured with an acetate seal incubated for 15 min at RT, protected from light. Stop Solution was then added to each well and the plates shaken gently to mix the solution. Absorbance was read at 450 nm within 15 min of addition of the Stop Solution using a Varioskan Lux plate reader (ThermoFisher Scientific, Carlsbad, CA). Relative optical densities (ODs) were background-corrected against blank samples and standard curves. T4 concentrations were interpolated using the four-parameter curve-fit method. Unknown sample concentrations were determined using GraphPad Prism software (GraphPad Prism 9.0.2, GraphPad Software, San Diego, CA).

### Data Analysis

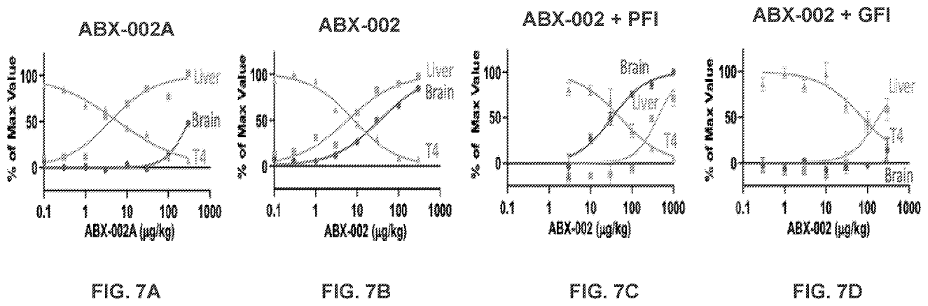

FIGS. 7A, 7B, 7C and 7D show gene expression in brain (blue) & liver (orange), & effects on T4 (gray) 4 or 8 h after last dose in mice that had been dosed PO, QD for 7 days with (A) active metabolite or (B) prodrug alone; (C) prodrug+PFI (LL-650177) or (D) prodrug+GFI. Both prodrug and active metabolite reduce T4 levels after 7 days of treatment. Table 6 reports ED₅₀ values in μg/kg for each treatment type.

TABLE 6

| | Brain | Liver | T₄ |
|---|---|---|---|
| Treatment | (ED₅₀ mg/kg) | (ED₅₀ mg/kg) | (ED₅₀ mg/kg) |
| ABX-002A | 310 | 3.8 | 6.0 |
| ABX-002 | 44 | 2.5 | 3.4 |
| (n = 3) | | | |
| +LL- | 30 | 260 | 48 |
| 650177 | | | |
| +PF- | >300 | 124 | 89 |
| 04457845 | | | |

Using T₄ as a marker for effects on THA; T₄ parallels peripheral activity more than CNS activation of target genes. Negative regulation of T₄ by thyromimetics does not appear to be predominantly centrally-mediated because the effects on THA and liver gene expression parallel plasma distribution more closely than exposure or activity in the CNS, suggesting a primarily pituitary-driven effect. The combination of a thyromimetic prodrug and a PFI may further enhance delivery of thyromimetics to the brain and maximize centrally-targeted distribution.

### Example 10: Phase II Study—Evaluation of the Efficacy and Safety of ABX-002 in the Adjunctive Treatment of Major Depressive Disorder (MDD) in Adults

This is a Phase 2 randomized, double-blind, placebo-controlled study of the efficacy, safety, PK and pharmacodynamics of daily oral doses of ABX-002 or placebo in adult subjects with MDD.

The study includes 3 periods: a Screening Period of up to 28 days, a 28-day Treatment Period, and an 8-week Observation Period.

28-Day Screening Period—At the Screening visit, subjects will be assessed for inclusion/exclusion criteria. Subjects who meet all inclusion and exclusion criteria will be randomized 1:1 according to a computer-generated randomization code to a single oral daily dose of ABX-002 or placebo for 28 days in addition to their continued standard-of-care serotonergic antidepressant.

28-Day Treatment Period—During treatment, subjects will be periodically assessed for efficacy, safety, and PK. An EEG and slit lamp examination will be obtained at the end of the Treatment Period. An EEG and slit lamp examination will be obtained at the end of the Treatment Period. After the Day 28 primary timepoint, all subjects will continue treatment with their background SSRI/SNRI.

Observation Period—Subjects responding to treatment will enter an 8-week Observation Period. A follow up exam will be conducted 14 days after the end of the Treatment Period. A slit lamp exam will be performed 12 weeks after the end of the Treatment Period. If the subject and Investigator elect to change the MDD treatment during the Observation Period, subjects will be followed for safety, but further efficacy data will not be collected. During the Observation Period, all AEs, regardless of causality, will be reported for 14 days following the last dose of study medication. Beyond 14 days, AEs should be reported only if they relate to the Eye Disorders System Organ Class in MedDRA 24.

Study treatment (ASK1 inhibitor or placebo) will consist of ABX-002 capsules or matching placebo, administered orally once daily.

The primary endpoint for the evaluation of efficacy is change from baseline to Day 28 in MADRS score. A determination of efficacy will be based on a 2-sided p-value <0.05. Secondary endpoints include change from baseline to Day 28 in Sheehan Disability Scale (SDS) score, change in CGI-S, change in SDQ (and the Energy subscale), change in C—SSRS, change in HAM-D, and the proportion of MADRS responders (≥50% reduction from baseline) and MADRS remission (≤9 at Day 28). The change from baseline in MADRS at Day 28 will be evaluated using a mixed model for repeated measures (MMRM). The model will include fixed effect for treatment group, visit, treatment-by-visit interaction, and baseline MADRS score.

### Inclusion Criteria:

Capable of giving informed consent and HIPAA authorization
Age 18-65 years at the Screening visit.
Able to understand the nature of the trial and, in the opinion of the Investigator, will follow protocol requirements.
DSM-5 primary diagnosis of a MDE as part of MDD by SCID-5-CV AND meets SAFER criteria (SAFER questions 1-4 must be scored as “Definitely” or “Possibly,” and SAFER questions 5-8 must be scored as “Definitely”).
Hamilton Depression Rating Scale (HAM-D) ≥19 at screening and at baseline. The HAM-D scores at baseline visit must be no more than 25% lower than at screening visit. Montgomery-Asberg Depression Rating Scale (MADRS) score ≥24 at baseline.
Clinical Global Impression of Severity (CGI-S) of moderately ill or worse at screening and at baseline prior to randomization.
Within the MDE, the subject is compliantly using a single SSRI or SNRI antidepressant for at least 6 weeks, with an adequate dose, and with an inadequate response as defined by the Antidepressant Treatment Response Questionnaire (ATRQ). In the MDE, at least 1 and no more than 2 antidepressants have been adequately assessed with an inadequate response as defined by ATRQ. The dosage of the current SSRI/SNRI must have been stable for the past 4 weeks and is expected to remain stable through screening and during the Treatment Period of this study.
Investigator and subject are willing to continue the existing SSRI/SNRI during the Treatment Period of this study. Investigator and subject are willing to continue the existing SSRI/SNRI during the Observation Period if the subject responds to treatment.
The subject should have detectable plasma levels of the SSRI/SNRI in a sample obtained during Screening.
Women must have a negative serum pregnancy test at Screening and before randomization at Day 1.
Women of childbearing potential and all male subjects must practice effective contraception during the study and for 90 days after the last dose of study drug.
BMI 18-35 kg/m2 inclusive and minimum weight of 50 kg.

### Exclusion Criteria:

History of schizophrenia or other psychotic disorder, or bipolar I or II disorder, delirium, dementia, amnestic disorder, or cognitive disorder. Current treatment for obsessive-convulsive disorder (OCD), attention deficit hyperactivity disorder (ADHD), posttraumatic stress disorder (PTSD), panic disorder, eating disorder, according to DSM-5 criteria.
Primary diagnosis of borderline, antisocial, paranoid, schizoid, schizotypal or histrionic personality disorder, according to DSM-5 criteria.
History of non-response to triiodothyronine (Cytomel® or T3) augmentation of depression.
Concurrent use of other augmentation agents for depression including second generation antipsychotics (SGA), lithium, bupropion or a second SSRI/SNRI.
Discontinued a second generation antipsychotics (SGA) for augmentation of MDD for lack of a treatment response may be screened for eligibility if the SGA was discontinued at least 30 days prior to Screening.
Received repetitive transcranial magnetic stimulation (rTMS) within 12 months of Screening or plan to receive rTMS during this study.
Received >1 course of electroconvulsive therapy (ECT) in their lifetime or plan to receive ECT during the study. History of vagal nerve stimulation (VNS) or deep brain stimulation (DBS).
History of alcohol or illicit substance abuse/dependence within 12 months of Screening (except for the use of caffeine or nicotine) or positive urine drug results at Screening or Baseline for an illicit substance. A single positive urine drug screening result for an illicit substance may be repeated once.
Actively suicidal (including, on the Columbia Suicide Severity Rating Scale [C—SSRS] Screening Version, an answer of YES to question 4 or 5 [current or over the last 6 months]) or has attempted suicide in the 2 years prior to Screening.
New psychotherapy or a change in the intensity of psychotherapy within 8 weeks before Screening. Psychotherapy, if present, must be expected to remain at the same intensity through Screening and the 28-day Treatment Period of this study.
Housing instability, receiving SS Disability, or legally incapacitated.
History or current evidence within previous 3 months before Screening of a serious or unstable neurologic, cardiovascular, gastrointestinal, respiratory, renal, hepatic, hematologic, endocrinologic, or other medical disorder, including cancer that would jeopardize the safe participation of the subject in the study (in the opinion of the Investigator).
History of prior MI, coronary artery balloon angioplasty or stent placement, heart failure, angina, or atrial fibrillation, atrial flutter, or ventricular tachycardia.
Clinically significant abnormality on ECG at Screening—males with QTc >450 msec and females with QTc >470 msec. One repeat ECG may be conducted and if the values are below these values, the subject may be included.
History of positive hepatitis B, Hep C virus (HCV) or human immunodeficiency virus (HIV) test.
Glycosylated hemoglobin (HbA1c) >8% at Screening.
Estimated creatinine clearance of ≤90 mL/min based on CKD-EPI equation.
Abnormal thyroid function tests at Screening (e.g., TSH, T3, TT4 and FT4). Or thyroid hormone supplementation (eg, history of treated hypothyroidism or thyroid carcinoma).
AST or ALT>2×ULN.
Lens opacity based on ophthalmological examination during Screening.
History of presence of clinically significant cataract, glaucoma, inflammatory eye disease or prior ophthalmic surgical procedures or laser surgery in either eye. Subjects with prior LASIK surgery may be included.
Evidence of epileptiform activity on EEG during Screening.
Diagnosis of epilepsy or history of convulsion, including childhood febrile seizure. Use of co-administered drugs that may lower seizure threshold is excluded (eg, bupropion).
Participated in any clinical research study within 30 days prior to Screening.
Using benzodiazepine hypnotics. Subjects with daily use of non-benzodiazepine hypnotics (eg, zolpidem, zaleplon, eszopiclone) may continue to use them during the 28-day Treatment Period as long as the dose is unchanged.
Known hypersensitivity to ABX-002 or its excipients.
Females who are pregnant, intend to become pregnant within 90 days of the last dose, or are breastfeeding.
Subjects who have previously participated in a clinical trial with ABX-002.
Investigative site personnel or their immediate families (spouse, parent, biological or adopted child or sibling).

What is claimed is:

1. A method of treating depression, anxiety disorders, or pain in a patient in need thereof comprising administering to the patient a therapeutically effective amount of a compound of Formula (I), or a pharmaceutically acceptable salt or solvate thereof: 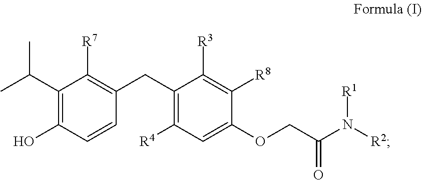
wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I;
R⁵ and R⁶ are independently selected from hydrogen and C₁-C₆alkyl; and
R⁷ and R⁸ are independently selected from hydrogen, —F, —Cl, —Br, and —I.

2. A method of treating depression, anxiety disorders, or pain in a patient in need thereof comprising administering to the patient a therapeutically effective amount of a compound of Formula (II), or a pharmaceutically acceptable salt or solvate thereof: 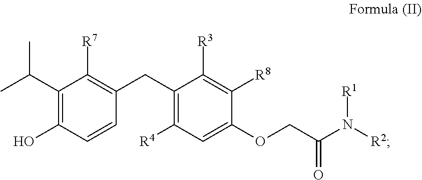
wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I;
R⁵ and R⁶ are independently selected from hydrogen and C₁-C₆alkyl; and
R⁷ and R⁸ are independently selected from hydrogen, —F, —Cl, —Br, and —I, wherein at least one of R⁷ and R⁸ is not hydrogen.

3. The method of claim 1 or 2, wherein R⁷ is hydrogen.

4. The method of claim 1 or 2, wherein R⁸ is hydrogen.

5. The method of any one of claims 1-3, wherein R⁸ is —F.

6. The method of any one of claims 1, 2, and 4, wherein R⁷ is —F.

7. A method of treating depression, anxiety disorders, or pain in a patient in need thereof comprising administering to the patient a therapeutically effective amount of a compound of Formula (III), or a pharmaceutically acceptable salt or solvate thereof: 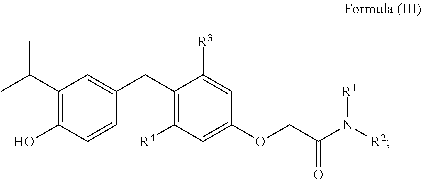
wherein:
R¹ and R² are independently selected from hydrogen, —OR⁵, —NR⁵R⁶, C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl, wherein C₁-C₆alkyl, C₂-C₆alkenyl, C₂-C₆alkynyl, C₃-C₆cycloalkyl, C₃-C₆heterocycloalkyl, phenyl, and —C₁-C₆alkyl-phenyl are optionally substituted with one or more of halo, cyano, —OR⁵, —NR⁵R⁶, —S(O)₂R⁵, or —S(O)₂OR⁵;
R³ and R⁴ are independently selected from —F, —Cl, —Br, and —I; and
R⁵ and R⁶ are independently selected from hydrogen and C₁-C₆alkyl.

8. The method of any one of claims 1-7, wherein R¹ is hydrogen.

9. The method of any one of claims 1-8, R² is C₁-C₆alkyl optionally substituted with one or more of halo, cyano, —OR⁵, —NR⁵R⁶, —S(O)₂R⁵, or —S(O)₂OR⁵.

10. The method of any one of claims 1-9, wherein R² is C₁-C₆alkyl substituted with one or more of halo, cyano, —OR⁵, —NR⁵R⁶, —S(O)₂R⁵, or —S(O)₂OR⁵.

11. The method of any one of claims 1-10, wherein R² is C₁-C₆alkyl substituted with one or more —OH.

12. The method of any one of claims 1-10, wherein R² is C₁-C₆alkyl substituted with one or more of halo.

13. The method of any one of claims 1-9, wherein R² is unsubstituted C₁-C₆alkyl.

14. The method of any one of claims 1-8, wherein R² is phenyl optionally substituted with one or more of halo, cyano, —OR⁵, —NR⁵R⁶, —S(O)₂R⁵, or —S(O)₂OR⁵.

15. The method of any one of claims 1-8, wherein R² is —C₁-C₆alkyl-phenyl optionally substituted with one or more of halo, cyano, —OR⁵, —NR⁵R⁶, —S(O)₂R⁵, or —S(O)₂OR⁵.

16. The method of any one of claims 1-15, wherein R³ and R⁴ are independently selected from —F, —Cl, —Br.

17. The method of any one of claims 1-16, wherein R³ and R⁴ are both —Br.

18. The method of any one of claims 1-16, wherein R³ and R⁴ are both —Cl.

19. The method of any one of claims 1-16, wherein R³ and R⁴ are both —F.

20. A method of treating depression, anxiety disorders, or pain in a patient in need thereof comprising administering to the patient a therapeutically effective amount of a compound, or a pharmaceutically acceptable salt or solvate thereof, selected from: 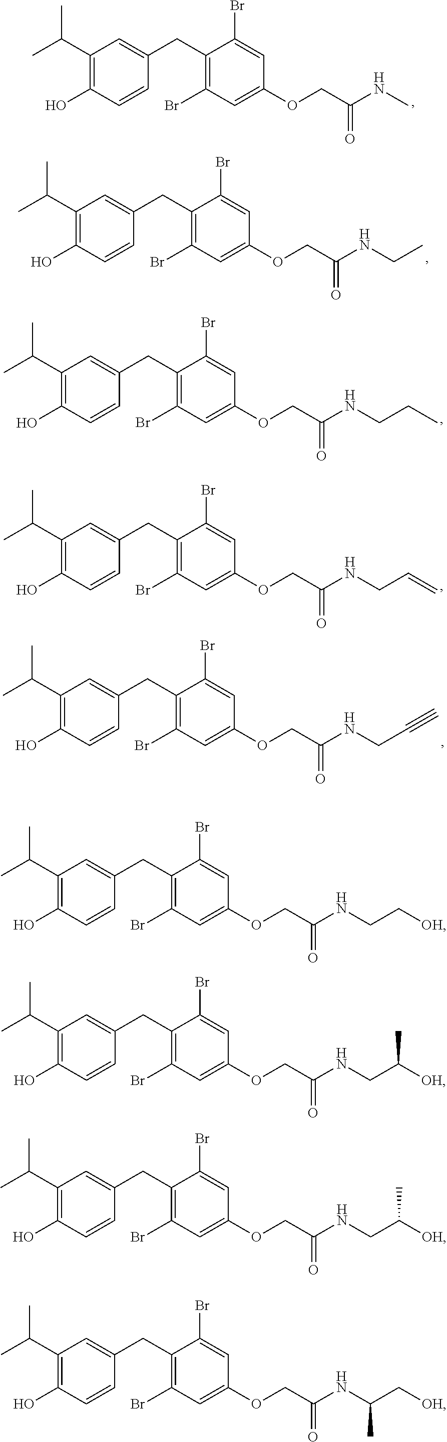 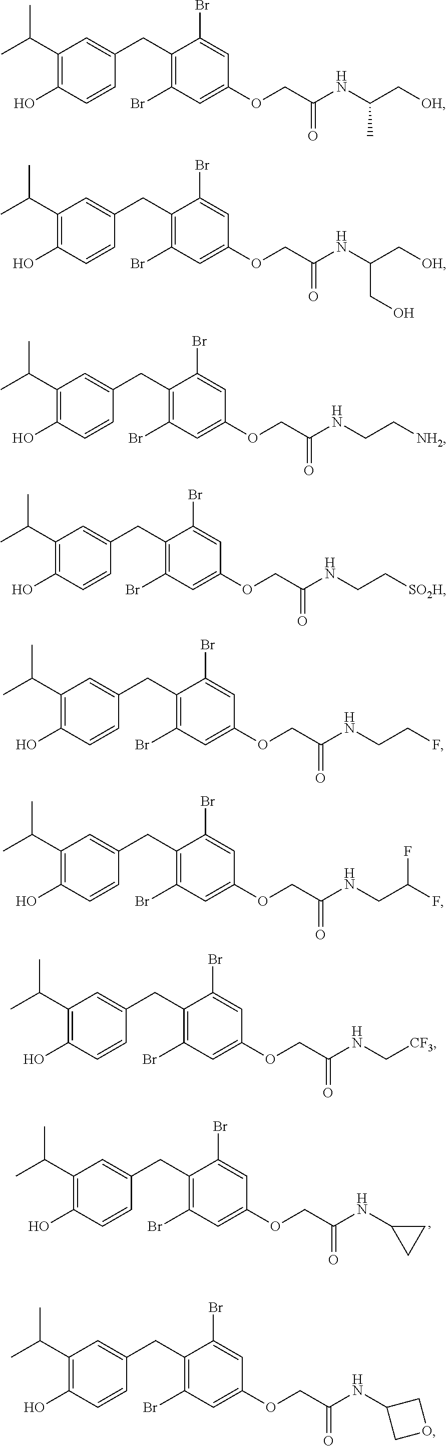 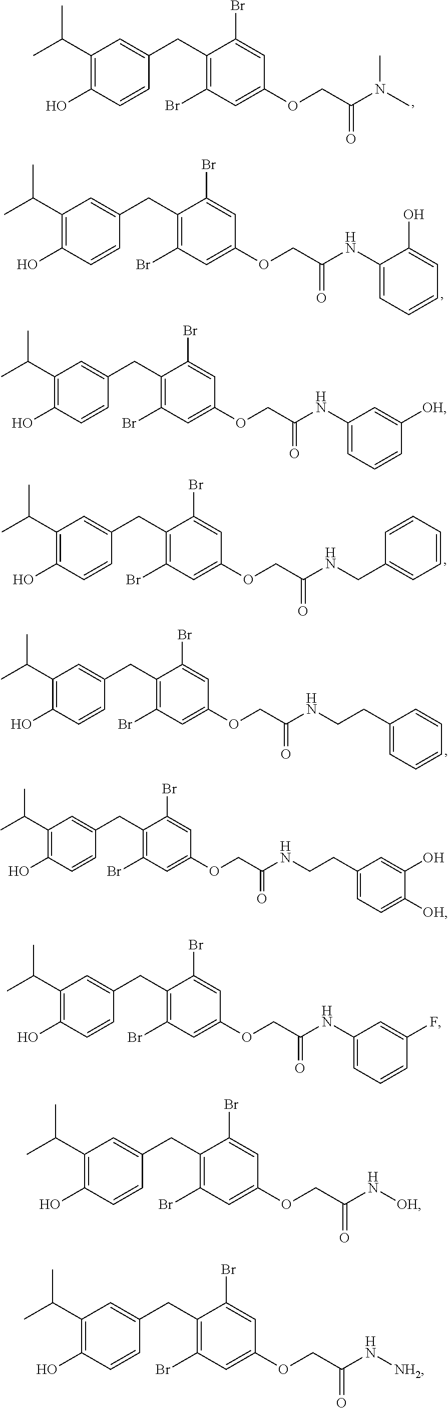 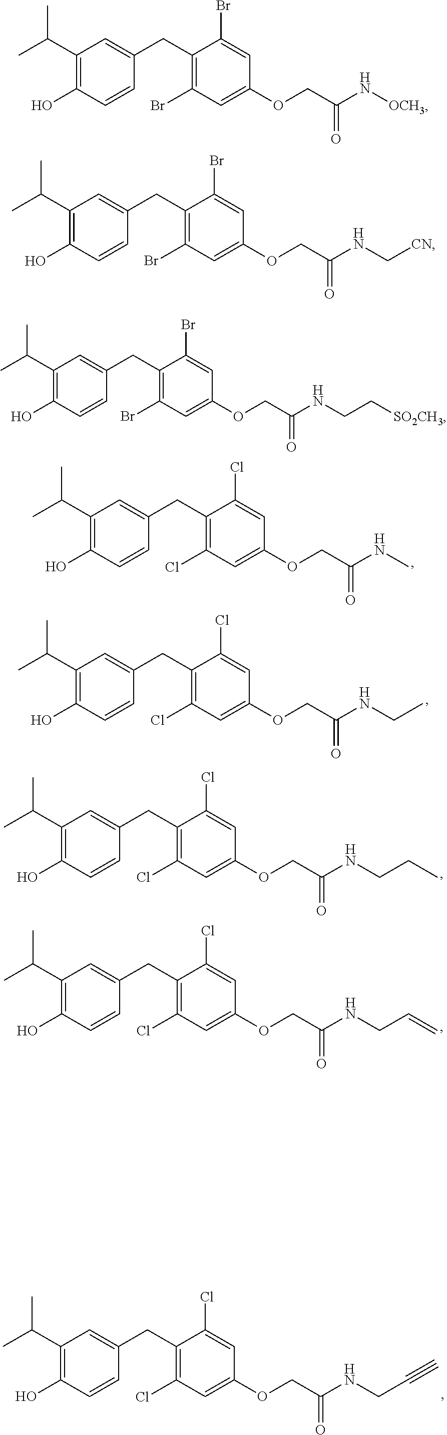 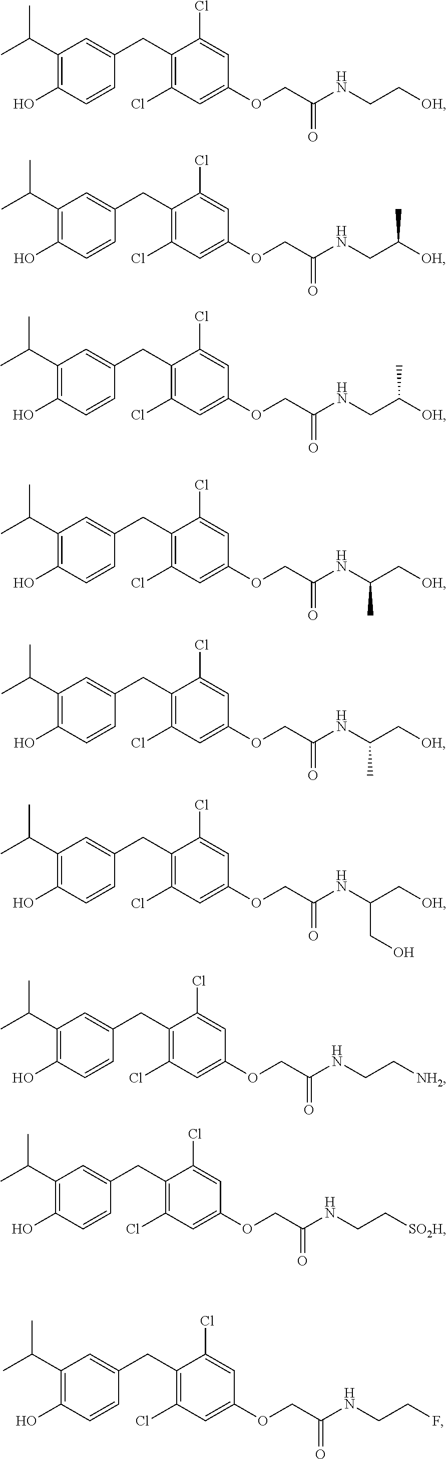 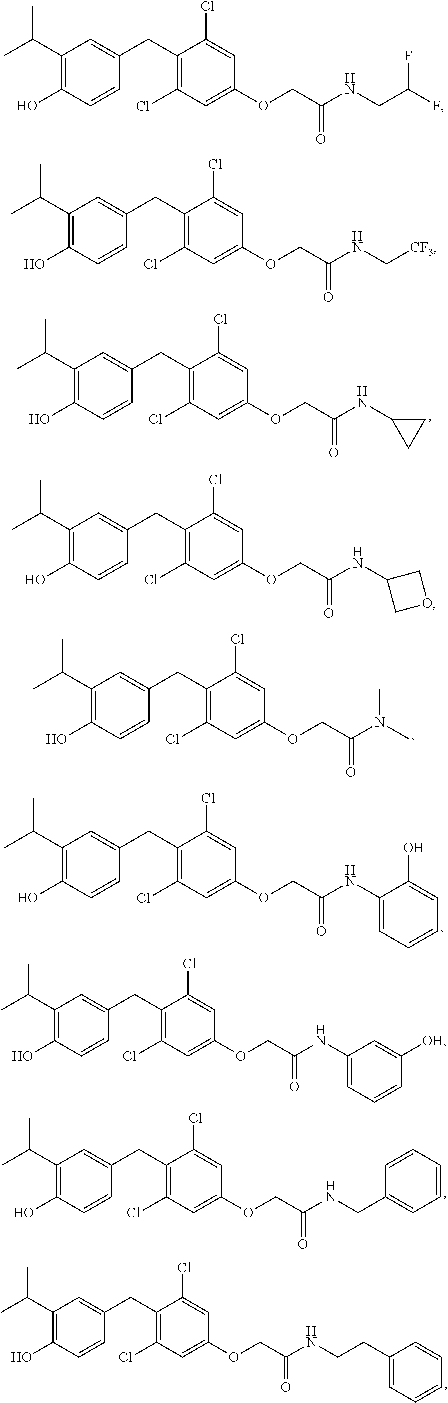 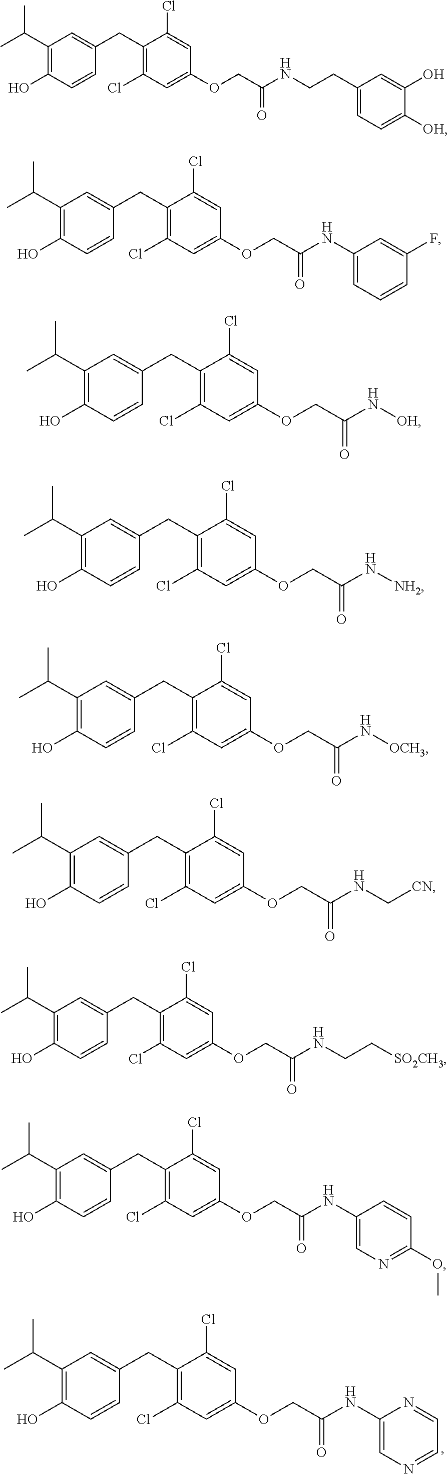 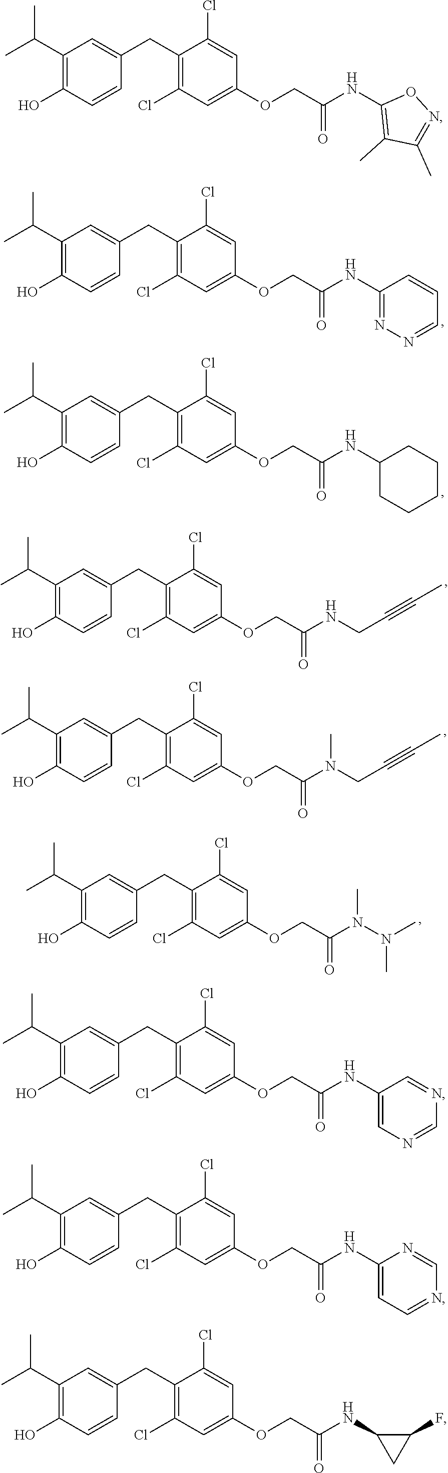 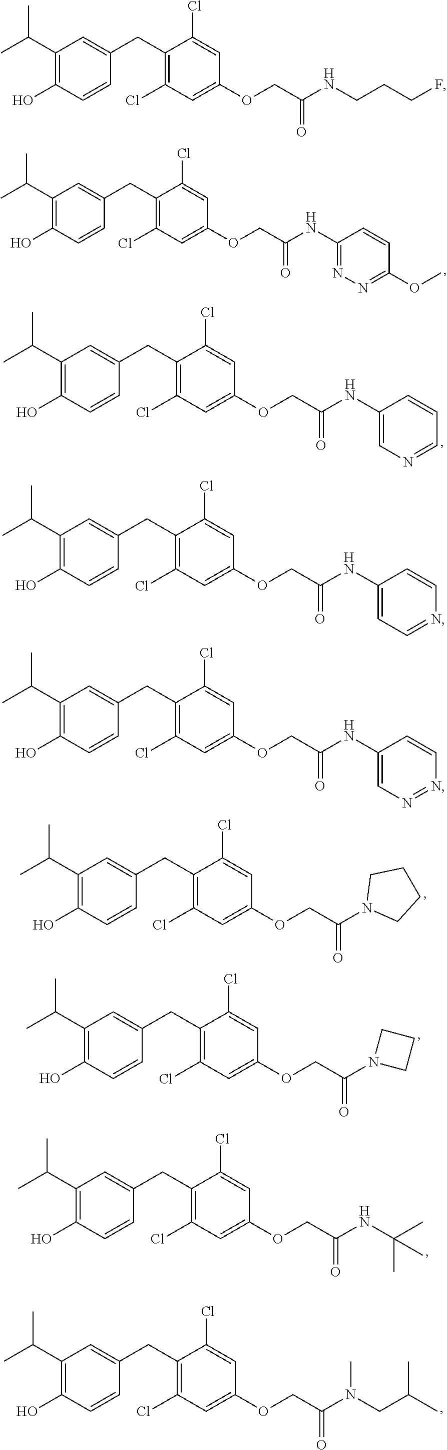 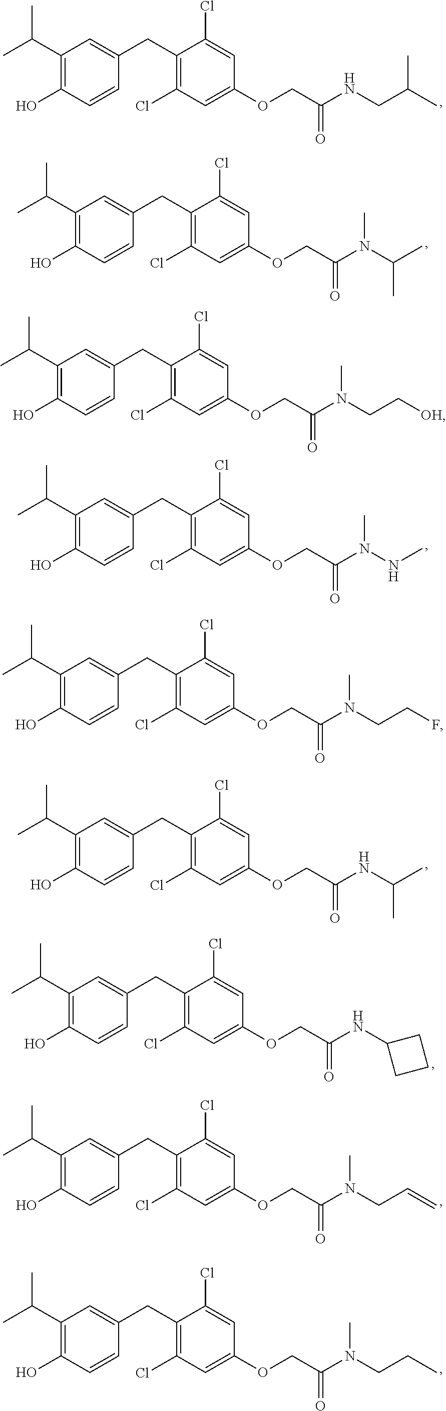 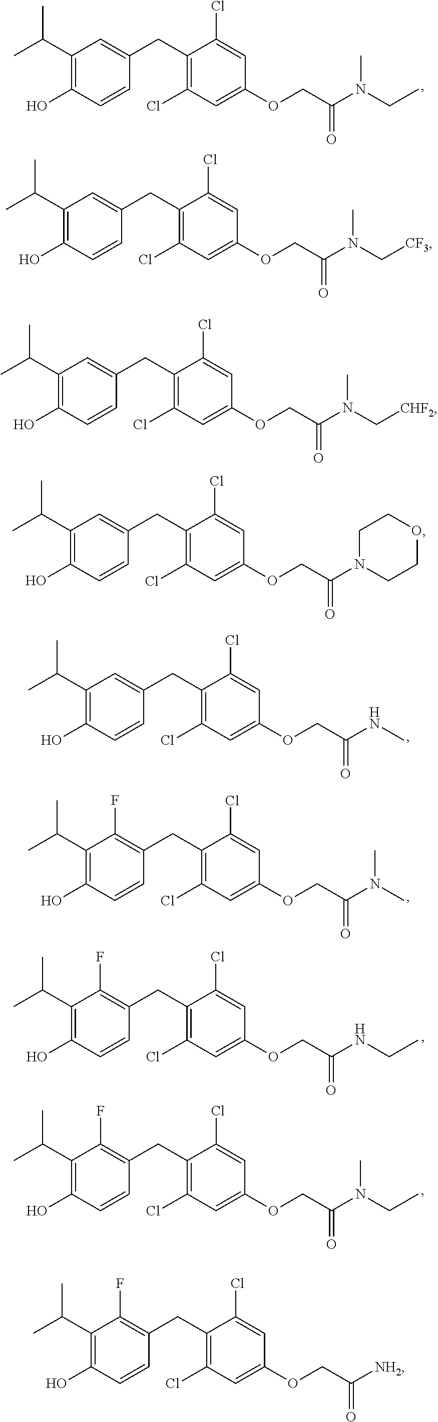 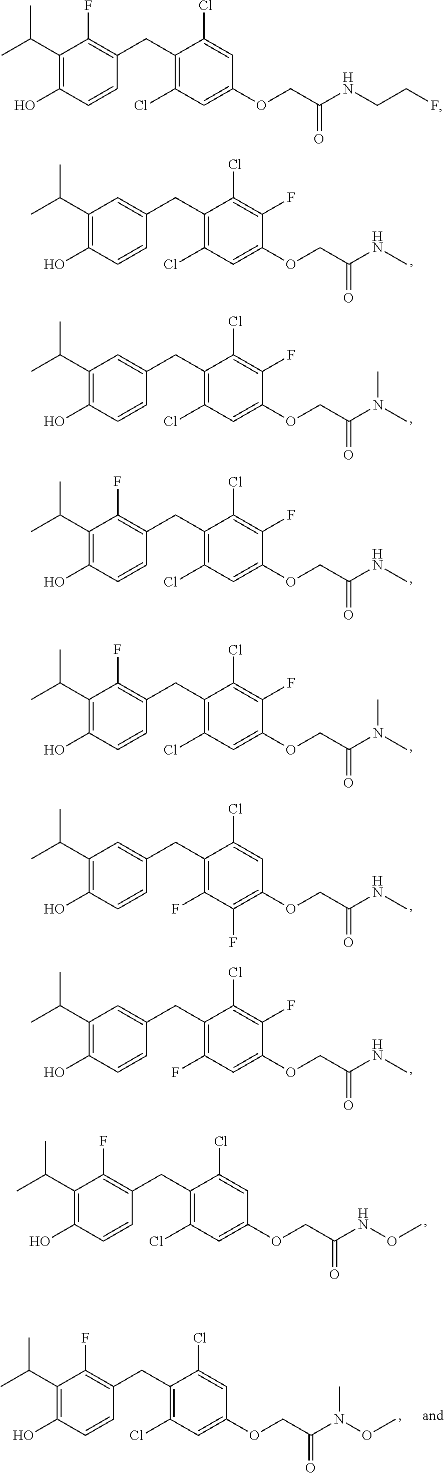 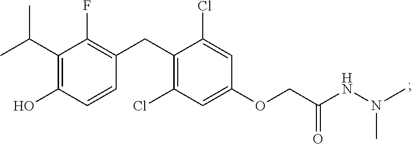 [SMILES: CC(C)c1c(O)ccc(Cc2c(Cl)cc(OCC(=O)NN(C)C)cc2Cl)c1F]
or a pharmaceutically acceptable salt or solvate thereof.

21. The method of any one of claims 1-20, for treating depression in a patient in need thereof.

22. The method of any one of claims 1-21, wherein the depression is major depressive disorder, treatment-resistant depression, seasonal affective disorder, psychotic depression, postpartum depression, melancholic depression, atypical depression, or catatonic depression.

23. The method of any one of claims 1-21, wherein the depression is bipolar depression, bipolar treatment-resistant depression, disruptive mood dysregulation disorder, persistent depressive disorder, depressed mood, premenstrual dysphoric disorder, medication-induced depressive disorder, postpartum depression, perimenopausal depression, multi-infarct dementia with depression, presenile dementia with depression, senile dementia with depression, vascular dementia with depressed moods, vascular dementia with depression, or unspecified depressive disorder.

24. The method of any one of claims 1-20, for treating an anxiety disorder in a patient in need thereof.

25. The method of claim 24, wherein the anxiety disorder is obsessive compulsive disorder, post-traumatic stress disorder, or a severe phobia.

26. The method of claim 25, wherein the severe phobia is agoraphobia or social phobia.

27. The method of any one of claims 1-20, for treating pain in a patient in need thereof.

28. The method of claim 27, wherein the pain is selected from migraine pain, chronic pain, chronic nerve pain, chronic muscle pain, chronic joint pain, diabetic neuropathy, fibromyalgia, back pain, and osteoarthritis pain.

29. The method of any one of claims 1-28, further comprising administering to the patient a peripherally restricted FAAH inhibitor.

30. The method of claim 29, wherein the peripherally restricted FAAH inhibitor is ASP-3652.

31. The method of any one of claims 1-30, further comprising administering to the patient a selective serotonin reuptake inhibitor (SSRI) or a serotonin and norepinephrine reuptake inhibitor (SRNI).

32. The method of claim 31, wherein the selective serotonin reuptake inhibitor (SSRI) is citalopram, escitalopram, fluoxetine, fluvoxamine, paroxetine, or sertraline.

33. The method of claim 31, wherein the serotonin and norepinephrine reuptake inhibitor (SRNI) is desvenlafaxine, duloxetine, levomilnacipran, milnacipran, sibutramine, tramadol, or venlafaxine.
